(19) United States
(12) Patent Application Publication
(10) Pub. No.: US 2025/0157612 A1
(43) Pub. Date: May 15, 2025

(54) METHOD AND SYSTEM FOR CONTROLLING THE DELIVERY OF ACTIVE AGENTS

(71) Applicant: Syqe Medical Ltd., Tel-Aviv (IL)

(72) Inventors: Perry DAVIDSON, Safed (IL); Binyamin SCHWARTZ, Sde Eliezer (IL); Aaron SCHORR, Lotem (IL); Yotam Eden HOFFMANN, Safed (IL)

(73) Assignee: Syqe Medical Ltd., Tel-Aviv (IL)

(21) Appl. No.: 19/026,873

(22) Filed: Jan. 17, 2025

Related U.S. Application Data
(63) Continuation of application No. 16/069,176, filed on Jul. 11, 2018.
(60) Provisional application No. 62/277,325, filed on Jan. 11, 2016.

(51) Int. Cl. G16H 20/10; A61M 11/04; A61M 15/00; A61M 16/00; G16H 20/13; G16H 50/70

(52) U.S. Cl. CPC G16H 20/10; A61M 15/0065; A61M 15/0066; G16H 20/13; G16H 50/70; A61M 11/042; A61M 15/0003; A61M 15/0048; A61M 15/0051; A61M 2016/0024; A61M 2205/13; A61M 2205/17; A61M 2205/18; A61M 2205/276; A61M 2205/332; A61M 2205/3368; A61M 2205/3375; A61M 2205/3553; A61M 2205/3584; A61M 2205/50; A61M 2205/505; A61M 2205/52; A61M 2205/6009; A61M 2205/6018; A61M 2205/6054; A61M 2205/6072; A61M 2205/609; A61M 2209/06; A61M 2230/06; A61M 2230/10; A61M 2230/30; A61M 2230/63

(57) ABSTRACT

Systems and methods for analyzing and/or using information collected from a plurality of users in conjunction with the use of delivery devices for active agents are described. In some embodiments, the delivery device comprises an inhaler device. In some embodiments, the information collecting comprises “crowd sourcing”, including collection of inputs from any one or more of a potentially wide variety of user-side data sources. In some embodiments, input collected from a plurality of users in a user population is used to adjust one or regimens for delivery of active agents to individual users. Optionally, regimen effect information comprising reports and/or pharmaceutical effect predictions is provided to individual users based on inputs collected from a plurality of users.

### RELATED APPLICATIONS

This application is a continuation of U.S. patent application Ser. No. 16/069,176 filed on Jul. 11, 2018, which is a National Phase of PCT Patent Application No. PCT/IL2017/050035 having International Filing Date of Jan. 11, 2017, which claims the benefit of priority under 35 USC § 119 (e) of U.S. Provisional Patent Application No. 62/277,325 filed on Jan. 11, 2016. The contents of the above applications are all incorporated by reference as it fully set forth herein in their entirety.

### FIELD AND BACKGROUND OF THE INVENTION

The present disclosure, in some embodiments thereof, relates to the field of substance administration; and more particularly, but not exclusively, to methods, devices and systems for controlled delivery of active pharmacological agents; for example, delivery by inhalation.

Natural substances, such as plant materials, have been used throughout history for medicinal and recreational purposes. Uses include the inhalation of fumes or vapors comprising active agents released from the natural substances. Such active agents can provide a wide range of pharmaceutical, therapeutic and other beneficial effects; as well as varying recreational effects, including sensory perception effects (scent and/or smell) and/or sought-after psychoactive effects. In addition, such substances can cause undesired side-effects; optionally desired or undesired, subject to circumstances.

One of the most used and studied natural substance is cannabis, which has been shown to have beneficial effects in treating nausea and vomiting, multiple sclerosis and other neurological conditions, loss of appetite and weight in cancer and AIDS, neurological pain, insomnia, anxiety and depression, epilepsy and other seizures, asthma, opioid withdrawal, and inhibition of primary tumor growth; as well as being effective in antipyretic and anti-inflammatory, antihelmintic, antimigraine and oxytocic applications. Cannabis has also been in wide use for recreational purposes, with various strains having different compositions of active agents and accordingly producing different desired effects.

International Patent Publication No. WO 2012/085919 to Davidson et al., discloses among other things an inhalation device for controlled extraction/vaporization of active agents from plant material by application of heat, wherein the plant material is organized as a cartridge and the device is configured to vaporize a precise amount of an agent in a highly reproducible manner. The inhaler comprises preloaded and pre-weighed plant material portions, each associated with a dedicated heating element designed to apply heat to the plant material matter to thereby vaporize one or more active agents from the plant material.

It is noted by the current inventors that using plant material as a source for active agents is often different than delivery of extracts of one or more of the active agents that are present in such plant material. This may be due to a combined effect of many components present in the plant material and absent from an extract and more so from a purified or synthetic agent. Such components may include one or more active agents as well as provide scent and/or taste effects. Additionally, it is noted by the current inventor that different strains of plants may contain different amounts and/or different ratios of active agents.

International Patent Publication No. WO 2016/001924 to Davidson et al. discloses inter alia methods and devices for controlling the pulmonary delivery of at least one active agent to a subject, wherein at least one pre-determined vaporized amount of the active agent is selected so as to achieve at least one pre-determined pharmacokinetic effect and/or at least one pre-determined pharmacodynamic effect induced by the agent in the subject. WO 2016/001924 further discloses methods and devices for adjusting the vaporized amount of the active agent based upon feedback from the subject.

International Patent Publication No. WO 2016/001922 to Davidson et al. discloses, among other things, methods and devices for pulmonary delivering to a subject at least two pharmacologically active agents, by providing different dose units comprising different active agents within the device and controlling the relative vaporization timing thereof so as to allow selective vaporization of the different active agents or active agents composition, each for different inhalation events (e.g., by selecting a dose unit having a desired composition and/or by matching a plurality of dose units to provide a desired amount/combination of active agents).

International Patent Publication No. WO 2016/001925 to Davidson et al. discloses, among other things, methods and devices for pulmonary delivering to a subject at least one pharmacologically active agents using simultaneously two or more different dose cartridges.

### SUMMARY OF THE INVENTION

There is provided, in accordance with some embodiments of the present disclosure, a system for dynamic definition of a regimen defining parameters for administration of at least one active agent to a user from a delivery device, the system comprising a server configured to: receive, over a digital communication network, input data indicating effects induced in a plurality of users each receiving administration of a regimen using a corresponding plurality of the delivery device; set at least one parameter of an administration regimen to a setting selected based on the received input data; and provide the adjusted administration regimen over the digital communication network to at least one delivery device for administration to at least one user.

According to some embodiments of the present disclosure, the system is configured to provide the adjusted administration regimen to the at least one user within a time period of year or less from receiving the input data on which parameter setting was based.

According to some embodiments of the present disclosure, the system is configured to provide the adjusted administration regimen to the at least one user within a time period of 1 month or less from receiving the input data on which parameter setting was based.

According to some embodiments of the present disclosure, the time period is shorter than the time period allotted for a treatment plan.

According to some embodiments of the present disclosure, the system is configured to provide an adjusted at least two adjusted administration regimens to the same user within a single year.

According to some embodiments of the present disclosure, the server is configured to set the at least one parameter by at least one of the group consisting of: adding one or more active agents and/or source materials to the administration regimen; removing one or more active agents and/or source materials from the administration regimen; increasing or decreasing an amount of at least one active agent delivered in the regimen; replacing at least one source material in the regimen; adding and/or removing at least one source material from the regimen; and modifying the composition of active agents delivered from a source material being used in the administration regimen.

According to some embodiments of the present disclosure, the indicated effects correlate with users individually modifying a regimen for their own subsequent use, and wherein the server sets the at least one parameter of the administration regimen according to a trend in the user modifications.

According to some embodiments of the present disclosure, the server is configured to set the at least one parameter of the administration regimen based on effects induced in a portion of the plurality of users, wherein the portion of the plurality of users is selected from the total plurality of users based on similarity of at least one of their characteristics to at least one characteristic of the at least one user.

According to some embodiments of the present disclosure, the similarity of at least one characteristic comprises similar susceptibility to effects produced by the administration regimen.

According to some embodiments of the present disclosure, the susceptibility is to side-effects produced by the administration regimen.

According to some embodiments of the present disclosure, the susceptibility is to targeted effects produced by the administration regimen.

According to some embodiments of the present disclosure, the similarity of characteristics comprises similarity of at least one of the group consisting of age, sex, weight, and daily level of activity.

According to some embodiments of the present disclosure, the server is configured to set the at least one parameter of the administration regimen using an implementation of a machine learning algorithm.

According to some embodiments of the present disclosure, the regimen comprises a regimen producing one or more of recreational effects and cognitive altering effects in the at least one user.

According to some embodiments of the present disclosure, the regimen comprises at least one parameter which is defined according to one or more characteristics of individual users, and wherein the server sets the at least one parameter of the administration regimen so that the definition of the parameter from the characteristics of individual users is changed.

According to some embodiments of the present disclosure, at least one of the parameters of the regimen is defined by a range of settings, wherein the plurality of users receive administration of the regimen using arbitrary settings selected from the range, and wherein the server sets the at least one parameter of the regimen based on differences in the indicated effects correlated with differences in the arbitrary settings.

According to some embodiments of the present disclosure, the server sets the at least one parameter of the administration regimen based on indicated effects that are known to be related to a setting of the at least one parameter.

According to some embodiments of the present disclosure, at least one parameter of the regimen comprises an amount of an active agent delivered by the regimen.

According to some embodiments of the present disclosure, the regimen defines parameters for the treatment of at least one of the diseases in the group consisting of: central nervous system diseases, a parkinsonism disorder, multiple sclerosis, autism, and obsessive compulsive disorder.

According to some embodiments of the present disclosure, the at least one active agent is extracted by the delivery device from botanical material.

According to some embodiments of the present disclosure, the administration regimen defines use of a combination of differing botanical materials used to treat a particular condition.

According to some embodiments of the present disclosure, the administration regimen defines dosages of differing botanical materials used to treat a particular condition.

According to some embodiments of the present disclosure, the input data includes information relating to user symptoms.

According to some embodiments of the present disclosure, the setting selected based on the received input data is also selected based on input data indicative of symptoms of the at least one user.

According to some embodiments of the present disclosure, the input data includes information relating to genetic information.

According to some embodiments of the present disclosure, the input data is received for a plurality of administrations of regimens to at least a portion of the plurality of users, and includes information about how dosing of each user of the portion has changed over time.

According to some embodiments of the present disclosure, the input data includes medical history information about at least some of the plurality of users.

According to some embodiments of the present disclosure, the regimen defines at least one of the group consisting of: an active substance, a type of botanical material used, a dosage amount, a frequency of use, a duration of use, and a timing of use.

According to some embodiments of the present disclosure, the input data includes at least one of the group consisting of: past dosage amounts, past frequency of use, past duration of use, past timing of use, and an effect related to pharmacodynamics or pharmacokinetics observed during the use of the delivery devices.

According to some embodiments of the present disclosure, the input data includes user preferences.

According to some embodiments of the present disclosure, the input data includes user status recognition by at least one of facial and vocal expression.

According to some embodiments of the present disclosure, the active agent is delivered from at least one botanical material, and the input data includes at least one from among the group consisting of: source plant species, source plant variety, source plant growing technique, nutrition factors during source plant growth, lighting conditions during source plant growth, composition of soil used to grow the source plant, active agent composition of the botanical material, and method used to prepare the botanical.

According to some embodiments of the present disclosure, the input data includes output from the delivery device in reference to each dose intake, comprising at least one of the group consisting of: sensed temperature, sensed pressure, sensed flow, sensed humidity, sensed location, device hardware version, and device software version.

According to some embodiments of the present disclosure, the server is furthermore configured to reject input data which indicates a potential attempt by a user to provide input data manipulating the system to produce an administration regimen which will interfere with achieving targeted effects of the administration to the at least one user.

According to some embodiments of the present disclosure, the delivery device is an inhaler.

According to some embodiments of the present disclosure, operating the inhaler to administer the administration regimen comprises extracting at least one active agent from botanical material.

According to some embodiments of the present disclosure, the input data comprise an indication of production lots of cartridges comprising material containing the active agent delivered by the delivery device, and wherein the server sets the at least one parameter of the administration regimen to correct for differences in effects induced from cartridges in different production lots.

According to some embodiments of the present disclosure, the input data comprise an indication of storage conditions of cartridges comprising material containing the active agent delivered by the delivery device, and wherein the server sets the at least one parameter of the administration regimen to correct for differences in effects induced from cartridges which experienced different storage conditions.

According to some embodiments of the present disclosure, the indication of storage conditions comprises an indication of an extreme of temperature experienced by the cartridges.

According to some embodiments of the present disclosure, the indication of storage conditions comprises a time-sequence of recorded climate monitoring values.

There is provided, in accordance with some embodiments of the present disclosure, a method for dynamic definition of a regimen defining parameters for administration of at least one active agent to a user from a delivery device, the method comprising: receiving, over a digital communication network, input data indicating effects induced in a plurality of users each receiving administration of a regimen using a corresponding plurality of the delivery device; setting at least one parameter of an administration regimen to a setting selected based on the received input data; and providing the administration regimen over the digital communication network to at least one delivery device for administration to at least one user.

According to some embodiments of the present disclosure, the administration regimen comprises a regimen producing recreational effects in the at least one user.

According to some embodiments of the present disclosure, the effects indicated in the input data comprise recreational effects on the plurality of users.

According to some embodiments of the present disclosure, the administration regimen comprises a regimen producing a targeted cognitive effect in the at least one user.

According to some embodiments of the present disclosure, the targeted cognitive effect includes one or more of enhanced alertness, enhanced concentration, lower fatigue, increased stamina and reduced fear.

According to some embodiments of the present disclosure, the regimen consists of delivery of at least one agent from a single source material.

According to some embodiments of the present disclosure, the delivery device is an inhaler device, and the at least one parameter includes the number of inhalations from the single source material and one or more extraction parameters for at least one of the inhalations.

According to some embodiments of the present disclosure, the setting comprises adjusting a delivered amount of at least one active agent, wherein the delivered amount in the administration regimen is different than an amount delivered in a previous administration regimen to the at least one user, and the amount is changed by less than 200 mg.

There is provided, in accordance with some embodiments of the present disclosure, a system for detection of a potential for substance abuse by a user of a delivery device configured for administration of at least one active agent, the system comprising a server configured to: receive input data indicating delivery device use histories for a plurality of users of a plurality of delivery devices, wherein the input data include use history data of users previously identified as having transitioned to become a substance abuser; receive input data indicating delivery device use histories for at least one target user; identify at least one of the target users at potential risk for transitioning to become a substance abuser, based on similarity of the target user's use history to use histories of identified substance abusers, in periods before they became substance abusers.

There is provided, in accordance with some embodiments of the present disclosure, a method of providing feedback to a user of a delivery device configured for administration of at least one active agent, the method comprising: determining a time since a user initiated a regimen for delivery of the at least one active agent; and providing to the user a computer interface indication of a current predicted effect of the at least one active agent; wherein the provided indication is based on input data indicating effects induced in a plurality of users receiving administration of the regimen and the time of the effects since regimen initiation.

According to some embodiments of the present disclosure, the method comprises adjusting the provided indication, based on reports of effects provided by the user.

According to some embodiments of the present disclosure, the method comprises showing to the user information from the plurality of users indicating aspects of the effects.

According to some embodiments of the present disclosure, the information presented to the user is selected based on the time since the user initiated the regimen, and times since regimen initiation that the information was reported by the plurality of users.

There is provided, in accordance with some embodiments of the present disclosure, a system for adjusting an administration regimen based on data gathered during use across a population, the system comprising at least one server configured to: receive data associated with usage of a delivery device from a plurality of users receiving administration of a regimen using a corresponding plurality of the delivery device, wherein each device is configured to deliver regimen-selectable dosages of one or more active agents from one or more substances, and wherein each delivery device is configured with a data transmission device to transmit to the at least one server input obtained from the user; determine at least one trend in the received data, based on processing of the data received from the plurality of users; adjust the regimen based on the at least one trend; and transmit the adjusted regimen to at least one device for use by at least one user.

According to some embodiments of the present disclosure, the data transmission device comprises a network interface configured to transmit to the at least one server input obtained from the user.

According to some embodiments of the present disclosure, the delivery device comprises an auxiliary computer device comprising a network interface configured to transmit to the at least one server input obtained from the user.

According to some embodiments of the present disclosure, the one or more substances comprise a plurality of substances.

According to some embodiments of the present disclosure, the trend in the received data comprises a trend in how the delivery device is used to deliver the one or more active agents.

According to some embodiments of the present disclosure, the trend in the received data comprises a trend in a parameter of regimens used by the plurality of users.

According to some embodiments of the present disclosure, the plurality of devices includes a plurality of inhaler devices, and the regimen includes inhalation of at least one active agent using an inhaler device.

There is provided, in accordance with some embodiments of the present disclosure, a method for dynamic adjustment of a user interface used with a delivery device for administration to a user of a regimen defining parameters for administration of at least one active agent, the method comprising: iteratively, for each of a plurality of users using the delivery device and providing input data through different versions of a user interface used therewith: receiving, over a digital communication network, input data indicating effects induced in the users receiving administration of the regimen; setting at least one parameter of the regimen to a new setting selected based on the received input data; and providing the modified regimen over the digital communication network for new administration to the plurality of users; selecting, from among the different versions of the user interface, the version of user interface used to provide input data which relatively most affected positive changes to parameters of the regimen; and providing the selected user interface for use by a new user, based on the selecting.

There is provided, in accordance with some embodiments of the present disclosure, a method for selection of a regimen defining parameters for administration of at least one active agent, and used with a delivery device for administration to a user, the method comprising: receiving, over a digital communication network, input data indicating effects induced in a plurality of users receiving administration of a sequence of a plurality of candidate regimens using a delivery device; selecting one of the candidate regimens based on the received input data; and providing the selected regimen, based on the selecting, over the digital communication network for administration to at least one user.

According to some embodiments of the present disclosure, the plurality of candidate regimens is provided to each user during a period of at least 1 week.

There is provided, in accordance with some embodiments of the present disclosure, a delivery device for administration of at least one active agent to a user, the delivery device comprising: a controller configured to select delivery by the delivery device of one of: at least a first preparation of material comprising the at least one active agent; and at least a second preparation of material prepared as a placebo for the first preparation; wherein the selection made by the controller is unapparent to the user at least until after the delivery device delivers the preparation selected for delivery.

There is provided, in accordance with some embodiments of the present disclosure, a delivery device for administration of at least one active agent to a user, the delivery device comprising: a controller configured to receive a command from a user to administer a preparation comprising the at least one active agent to the user; and deliver a substitute preparation to the user, without generating an indication of the substitution to the user.

Unless otherwise defined, all technical and/or scientific terms used herein have the same meaning as commonly understood by one of ordinary skill in the art to which the disclosure pertains. Although methods and materials similar or equivalent to those described herein can be used in the practice or testing of embodiments of the disclosure, exemplary methods and/or materials are described below. In case of conflict, the patent specification, including definitions, will control. In addition, the materials, methods, and examples are illustrative only and are not intended to be necessarily limiting.

As will be appreciated by one skilled in the art, aspects of the present disclosure may be embodied as a system, method or computer program product. Accordingly, aspects of the present disclosure may take the form of an entirely hardware embodiment, an entirely software embodiment (including firmware, resident software, micro-code, etc.) or an embodiment combining software and hardware aspects that May all generally be referred to herein as a “circuit,” “module” or “system.” Furthermore, some embodiments of the present disclosure may take the form of a computer program product embodied in one or more computer readable medium(s) having computer readable program code embodied thereon. Implementation of the method and/or system of some embodiments of the disclosure can involve performing and/or completing selected tasks manually, automatically, or a combination thereof. Moreover, according to actual instrumentation and equipment of some embodiments of the method and/or system of the disclosure, several selected tasks could be implemented by hardware, by software or by firmware and/or by a combination thereof, e.g., using an operating system.

For example, hardware for performing selected tasks according to some embodiments of the disclosure could be implemented as a chip or a circuit. As software, selected tasks according to some embodiments of the disclosure could be implemented as a plurality of software instructions being executed by a computer using any suitable operating system. In an exemplary embodiment of the disclosure, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the disclosure. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present disclosure may be written in any combination of one or more programming languages, including an object oriented programming language such as Java, Smalltalk, C++ or the like and conventional procedural programming languages, such as the “C” programming language or similar programming languages including for example mobile application programming languages such as angularJS and HTML5. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present disclosure may be described below with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

### BRIEF DESCRIPTION OF THE SEVERAL VIEWS OF THE DRAWINGS

Some embodiments of the disclosure are herein described, by way of example only, with reference to the accompanying drawings. With specific reference now to the drawings in detail, it is stressed that the particulars shown are by way of example, and for purposes of illustrative discussion of embodiments of the disclosure. In this regard, the description taken with the drawings makes apparent to those skilled in the art how embodiments of the disclosure may be practiced.

In the drawings:

FIG. 1 is a schematic flowchart describing adjustment and assignment of a regimen for delivery of at least one active agent to a user, based on feedback information from one or more users, according to some embodiments of the present disclosure;

FIG. 2 schematically illustrates an example of data collection from a plurality of patients undertaking an inhaler-delivered treatment regimen for a condition, followed by output of recommended adjustments to treatment regimens, according to some embodiments of the present disclosure.

FIG. 3 schematically represents use of an autonomous, continuous, multi active agent, selective dose-discovery, -delivery, -adjustment and/or -optimization system, according to some embodiments of the present disclosure.

FIG. 4 schematically represents a system for adaptive operation of an inhaler based on inputs from a plurality of users, according to some embodiments of the present disclosure;

FIG. 5 schematically represents modes of user feedback data collection and modes of providing to a user inhaler with use guidance and/or indications, according to some embodiments of the present disclosure;

FIG. 6A schematically represents a status bar indicating a user's current (actual and/or expected) state for some effect of a delivered active agent relative to a baseline time course for that effect, according to some embodiments of the present disclosure;

FIGS. 6B-6E schematically represents an effect time course graph, including an effect region, according to some embodiments of the present disclosure;

FIG. 7 schematically represents a method for user-driven development of new regimens based on active design inputs from users, according to some embodiments of the present disclosure;

FIG. 8 schematically represents relationships of machine learning to other aspects of a system for adaptive operation of an inhaler to some of the inputs to and results of machine learning, according to some embodiments of the present disclosure;

FIG. 9 schematically represents aspects of the system of FIG. 4 which are optionally available for customization by users and/or suppliers, according to some embodiments of the present disclosure;

FIGS. 10A-10B schematically illustrate an inhaler device configured to receive a plurality of source material cartridges, according to some embodiments of the present disclosure;

FIGS. 11A-11B respectively show an isometric view (FIG. 11A) and a side view (FIG. 11B) of an arrangement of source material sections structured to provide for separately accessing each of the plurality of source material sections, according to some embodiments of the present disclosure;

FIGS. 12A-12B illustrate a slidable actuator configured for unblocking at least one airflow path associated with at least one source material section and/or for activating a heating element associated with the at least one source material section, according to some embodiments of the present disclosure;

FIGS. 13A-13D show various structural features of an actuator, for example as described in FIGS. 12A-12B, according to some embodiments of the present disclosure; and

FIGS. 14A-14B illustrate a camshaft mechanism for using a plurality of source material sections in a serial manner, according to some embodiments of the present disclosure.

### DESCRIPTION OF SPECIFIC EMBODIMENTS

The present disclosure, in some embodiments thereof, relates to the field of substance administration; and more particularly, but not exclusively, to methods, devices and systems for controlled delivery of active pharmacological agents; for example, delivery by inhalation.

### Overview

A broad aspect of some embodiments of the present disclosure relates to systems and methods for analyzing and/or using information collected from a plurality of users in conjunction with the use of delivery devices for active agents. As used herein, the term “active agent” means a compound, a polymer, a conjugate or a complex, or any combination thereof, which exerts and/or contributes to a physiological or psychological effect when administered to a subject. The active agent may have or contribute to a pharmaceutical effect, a sensory effect (e.g., smell and/or taste) a psychoactive effect and/or a somatic effect. The active agent may be of natural origin or synthetic. In the context of some embodiments of the present disclosure, the active agent is a naturally occurring agent found in a naturally occurring substance (e.g., a natural plant substance, as described herein), or a metabolite thereof. In some embodiments, the agent is extracted and/or purified from a natural source. The term active agent also encompasses, unless otherwise indicated, two or more agents.

In some embodiments, the delivery device comprises an inhaler device. Examples herein focus the example of an inhaler device; however, it should be understood that the concepts described herein with respect to the particular example of an inhaler device optionally apply to any other device for metered delivery of active agents, changed as necessary.

In some embodiments, the information collecting comprises an example of what sometimes is called “crowd sourcing”, including collection of inputs from any one or more of a potentially wide variety of user-side data sources. Optionally, these user-side data sources include any one more of sensor data, device operation data (e.g. using device sensors), visual and/or audio data, and/or passively, semi-passively, and/or actively user-derived inputs. After collecting data, in some embodiments, uses of the data comprise processing of the data (for example, to extract meaningful trends), and providing results of the processing to one or more recipients. Recipients may include, for example: users, administrators of the system, physicians supervising users of the system, manufacturers, suppliers, researchers, and/or regulators (being third party or associated with the device or system manufacturer).

An aspect of some embodiments of the present disclosure relates to the use of input collected from a plurality of users in a population to adjust one or more regimens for delivery of active agents to individual users. Optionally, the individual users are new users who were not included in the population. Optionally, the new users may become part of the population from which input is collected. In some embodiments, the individual users are users of the same population from which data was collected. In some embodiments, a regimen comprises a protocol describing delivery of pharmaceutically active agent from one or more predetermined doses of a designated formulation; supplied, for example, in the form cartridges and/or magazines comprising a plurality of cartridges and/or a plurality of magazines.

In some embodiments, the population comprises a plurality of individuals suffering from the same or similar condition or symptom, and the regimen is intended to treat and/or alleviate and/or prevent the condition and/or treatment. In some embodiments, the regimen is intended for recreational purposes and the population comprises a plurality of individuals interested in such recreational effect. Optionally, the population is a sub-population, which is further defined based on one or more personal traits or preferences, such as age, gender, weight, background and medical history, occupation, genetic information, activity and user preferences.

In some embodiments, adjustment of a regimen comprises a change to a fixed-protocol regimen (e.g., a regimen defining delivery of the amounts with the same timing for every user of the regimen). In some embodiments, the regimen is fixed, but “fuzzy”—e.g., defining regimen parameter ranges, from which actual values are arbitrarily (for example, randomly) or purposefully (e.g. based on user requests and/or personal needs and/or traits) selected and assigned to users and/or used to define an administered regimen variant. In some embodiments, the adjusted regimen comprises a parameterized regimen, e.g., a regimen which includes descriptions of variations to the actual delivery of an active agent according to certain parameters (each of which optionally comprises an administered regimen variant). In some embodiments, the parameters affecting variation are associated with particular user-provided and/or user-specific inputs. Optionally, data from a plurality of administered regimen variants are used in adjusting of a parent regimen, and/or administered variants thereof. In some embodiments, data from separate regimens are combined for use in adjusting of a regimen; for example, regimens that are related by their use of at least some of the same substances and/or substance amounts. Optionally, data from a plurality of administered regimen variants and/or data from separate regimens are combined for use in defining a new regimen.

Selection of a regimen adjustment, in some embodiments, comprises analysis of user-derived inputs from previous applications of the regimen, based upon which the adjusted regimen is created. The target of the adjustment, in some embodiments, is to increase, decrease, extend, shorten, re-balance, and/or otherwise modify effects on users of the regimen, as indicated in the user-derived inputs. In some embodiments, the adjustment target is implicit in the input data (e.g., a targeted value of some inputted metric is not achieved, or a known undesirable side effect is insufficiently managed). In some embodiments, the adjustment target is explicitly indicated by users, for example based on reporting.

In some embodiments, the adjustment is determined by the use of a machine learning implementation (herein, the term “AI”, or artificial intelligence, is used interchangeably as a term for an implementation of a machine learning algorithm). Optionally, the cycling time between regimen result inputs being received, adjustments applicable to future regimen administrations provided to users and optionally also receiving the next regimen result inputs again received is kept short—for example a few minutes or less, a few hours or less, a day or two or less, a week or less, a couple of weeks or less, a month or less, 6 months or less, a year or less or another longer, shorter or intermediate time. Optionally, the cycling time is shorter than the time period allotted for a temporary treatment (i.e. not chronic or long term use such as one month or less, 6 months or less or a year or less). Optionally the cycle is measured between the last user input that is included in determining the adjustment and the first transmission of an adjusted regimen based on the input. Optionally, this allows regimen adjustments to occur in small increments while still producing substantial effects over time. Small increment changes are potentially beneficial, e.g., for early detection of potential problems developing, and/or for maintaining approximate linearity in responses to changes and/or for reducing the hazard of an extreme physiological or psychological reaction and/or in order to safely detect toxicity levels. Optionally, the increments may be in the range of 10 mg or less per dose, 50 mg or less per dose, 100 mg or less per dose, 200 mg or less per dose or 500 mg or less per dose. Optionally, the increments may be in the range of 10 μg or less per dose, 50 μg or less per dose, 100 μg or less per dose, 200 μg or less per dose or 500 μg or less per dose. Optionally the increments are performed as a percentage of a maximal planned or a maximal permitted dose (e.g. due to potential side effects and/or health risks). In such event the increments may be 1% or less of the maximal amount, 5% or less of the maximal amount, 10% or less of the maximal amount or even 20% or less of the maximal amount. Optionally, the increments reduce and the dosage increases. Optionally, the increments are at an amount low enough that each increment in itself is not expected to cause a detectable effect in the individual user and/or for an individual application of a regimen. Effects which are undetectable in individual users and/or regimen applications are potentially detected in a larger sample population, e.g., detected by machine learning and/or statistical techniques. Optionally the increments are adjusted at least once a day, at least once a week, at least once in two weeks at least once a month, at least once every six months, or at least once a year. Optionally, the adjustments occur at a frequency that causes a regimen to be updated for a user within a period of a course of treatment of a user. Optionally, the doses are extracted directly from raw plant material. Optionally the device is an inhaler and the extraction is controlled electronically.

An aspect of some embodiments of the present disclosure relates to providing regimen effect information to individual users based on inputs collected from a plurality of users. In some embodiments, the regimen effect information comprises a predicted time-course of one or more effects, the prediction being based on previous inputs from users that indicate, e.g., onset, peak and/or termination of targeted effects, level of the effect (e.g. symptom relief) and/or side effects. The information may relate to a plurality of different effects. Optionally the information is adjusted according to specific traits of the user receiving it. The information may be displayed to an individual user, e.g., on a dedicated indicator, a computer device screen (for example mobile phone), or in any other way. In some embodiments, an individual user may provide additional inputs, whereby the time-course predicted from the general population may be transformed to better indicate to the individual user what to expect personally.

In some embodiments, information provided to an individual user comprises reports taken from the plurality of users, optionally indexed to regimen time. In some embodiments, the user sees these reports in predicted (optionally calibrated) synchrony with their own experience of the regimen. Such reports may help to reassure a user that their own experience of the regimen is normal (if matching), or encourage a user to seek corrective action (if not matching).

Optionally, a user or potential user of a regimen browses reports freely, arranged in a timeline or otherwise arranged (e.g., sorted by report content and/or popularity). This is of potential use in helping users decide on a protocol which suites their needs.

In some embodiments, reports shown to a user may be filtered to those which the user considers (or is expected to consider) most relevant; e.g., reports from users with similar personal and/or disease characteristics.

Optionally, the user provides input regarding a desired time-course of one or more effects, and the regimen is selected and/or adjusted to best match the request. Optionally, the user is provided with information regarding the time-courses of one or more effects of each of a plurality of applicable regimen and is permitted to select the one that is most preferable to the user.

An aspect of some embodiments of the present disclosure relates to use of natural language inputs (and in particular, spoken inputs) in order to provide commands relating to administration of a regimen.

In some embodiments, commands provided by a user are couched in terms of effects on their own symptoms and/or state, rather than as specific regimen parameters. For example, instead of commanding “administer 1 mg of CBD”, a user command is couched in terms of the effect they are trying to achieve, for example “reduce my nausea”. In some embodiments, natural language commands couched in terms of an explicit or implicit desired result are mapped to a particular regimen based on results of machine learning applied to a training set of commands.

In some embodiments, a brain activity mapping device/EEG device can be used to measure regimen effect and/or provide brain activity feedback in association with specific pharmaceutically active agents administrated.

An aspect of some embodiments of the present disclosure relates to use of natural language inputs (spoken or written inputs) in order to provide experience reports relating to administration of a regimen. Optionally, the natural language inputs are provided in addition to pre-prepared input options from which the user may select.

In some embodiments, a delivery device for active agents, and or a computing device functionally connected thereto, is configured to receive arbitrary textual and/or spoken language inputs from users relating to the user experience. Such inputs are optionally provided before or after, and particularly during the user experience resulting from administration of a regimen. In some embodiments of the disclosure, machine implemented speech-to-text recognition is applied to spoken inputs. Optionally, tokens expressed in the inputs (such as words, phrases, tags such as hashtags, emojis, emoticons, and the like) are provided as inputs to a machine learning implementation, configured to detect expression usage trends therein. In some embodiments, usage trends are treated as inputs which optionally affect regimen adjustments (e.g., increased mentions of a side-effect in conjunction with a particular phase of a regimen are converted into an increase for that phase of the regimen of an agent known to counteract that side-effect). In some embodiments, expression usage trends are used to help determine effect prediction timelines for a user (e.g., mentions of “feeling it now”, “kicking in” and the like are found to associate with effect onset, etc.)

An aspect of some embodiments of the present disclosure relates to the production of new regimens using developer kits comprising a suitable array of dose materials for supporting experimentation (e.g., cartridges and/or cartridge magazines, and the substances, such as raw botanical materials and/or purified, extracted and/or synthetic active agents, loaded into them.). In some embodiments, developer kits differ from dose material normal regimen delivery by including a wider range of botanical types (species and/or plant varieties and/or purified, extracted and/or synthetic active agents, for example), potencies (amounts of active agent or available combinations of active agents), and/or options for combination. In some embodiments, a developer kit includes one or more cartridges, each comprising a plurality of individually addressable dose sections. Provided with such cartridges, a regimen developer can experiment with different combinations of dose sections (within and/or between different cartridges), before arriving at a combination which is suitable for production as a simpler cartridge and/or magazine arrangement.

An aspect of some embodiments of the disclosure relates to the providing to a user-operated application (e.g., an app on a computing device, and/or a portion of a network-accessed portal site) the capability of being expanded in functionality by plugin modules of one or more types. In some embodiments, a plugin interface is made available for plugin modules providing new ways of selecting and/or defining regimen parameters of a user. In some embodiments, a plugin interface is made available for plugin modules providing new ways for users to report and/or assess their status, and/or effects of regimen administration. In some embodiments, a plugin interface is made available for plugin modules which operate to accompany the experience of receiving regimen administration, for example, formatting and/or controlling display reports data from other users, providing sensory outputs to accompany the experience, providing games or other distractions to engage the user while waiting for effects, etc.

As used herein, the term “regimen”, in some embodiments, designates at least one, and in some embodiments a plurality of pre-determined doses comprising defined amounts of at least one particular active agent (e.g. a pharmaceutically active agent). For example, a dose delivered from raw cannabis may comprise a plurality of active agents, including for example cannabinoids and terpenes, which may depend on the specific strain of cannabis used, its condition, the extraction protocol and whether or not additional agents (active or not) were added to the cannabis.

In embodiments where a regimen comprises a plurality of pre-determined doses, the doses are optionally delivered together (e.g., at least partially overlapping in time of delivery) and/or separated in time at any suitable interval. Different doses optionally comprise chemically different compounds and/or agents. Different doses optionally comprise different amounts of compounds and/or agents (extracted from the same or different amounts of source material). In some embodiments, a regimen comprises an administration event (e.g., one dosage, or a plurality of dosages delivered over a short time related to a single instance of recreational and/or symptom reducing use). In some embodiments, a regimen comprises administration over a course of treatment, such as a treatment plan. For example, the treatment is intended to last until a condition or symptom is reversed or eliminated. In some embodiments, the treatment plan may be used in order to prevent a condition or symptom and/or extend indefinitely, such as in management of a chronic condition. Adjustments of a regimen, described herein, are optionally adjustments to an ongoing (e.g., course of treatment type) regimen, and/or to a regimen defined for individual administration events.

As used herein, the term “cartridge”, in some embodiments, includes an amount of source material from which at least one active agent is provided, which can be individually addressed by the device. For example, it may be in the form of an independent cartridge or as a portion in a continuous substance (for example in a ribbon or larger cartridge) or a cartridge comprising a plurality of distinct sections of source material. In some embodiments, the active agent content of source material in the cartridge is predetermined, e.g., set during production to a predetermined and reproducible amount. In some embodiments, the portion of the active agent content that is released from the cartridge is metered; that is, controlled to comprise a particular amount of at least one active agent that is controlled by parameters of a regimen. In some embodiments, a cartridge itself comprises a plurality of addressable dose sections. Optionally, these sections are activated (e.g., heated for vaporization of active agent) individually, simultaneously (e.g., at least partially overlapping in time of vaporization), and/or in any suitable sequence within a single inhalation, or spread out over several inhalations.

In some embodiments, a cartridge includes and/or indicates and/or is associated with extraction parameters for source material, designed to cause delivery of one or more selected amounts of one or more active agents from the source material. In some embodiments, extraction parameters are read from the cartridge itself and/or from packaging or written material that is associated with the cartridge. Additionally or alternatively, the cartridge and/or an associated packaging or printed material provides an identifier which allows extraction parameters to be obtained from another source, for example, a portal. The identifier may be read for example by a reader device (such as in case of a barcode or RFID) or by a user who can communicate it to the system via user interface. This optionally allows remotely controlled revisioning of extraction parameters, e.g., to allow use with new device models, and/or for quality assurance purposes. For example, a cartridge or section of a cartridge may hold a predefined amount of plant material (e.g. cannabis) having in it an active agent that is to be delivered to the user as part of a regimen (e.g. THC or CBD). Different amounts of the active agent(s) may be delivered to the user as part of a regimen from this source material by controlling parameters of operation of the device. For example, extraction parameters (for example airflow through the source material, temperature of the source material and/or time period of extraction) may be controlled. As long as a dose defined in a regimen is within the range that can be selectively delivered from the source material, different regimens may be delivered using identical cartridges.

As used herein, the term “magazine”, in some embodiments, includes a plurality of distinct cartridges. Optionally a cartridge is loaded into an inhaler such that access is granted to each of the plurality of cartridges, based on device constraints. In the case of a continuous substance that spans a plurality of cartridges, the substance may be deemed a magazine. Optionally, a magazine may hold a plurality of smaller magazines (such as a plurality of continuous substance masses).

Before explaining at least one embodiment of the disclosure in detail, it is to be understood that the disclosure is not necessarily limited in its application to the details of construction and the arrangement of the components and/or methods set forth in the following description and/or illustrated in the drawings. The disclosure is capable of other embodiments or of being practiced or carried out in various ways.

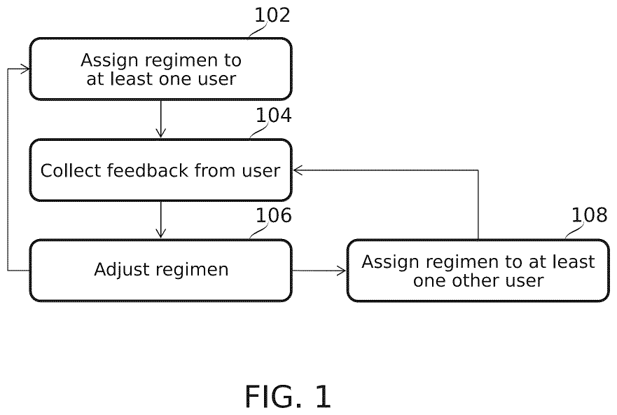

Reference is now made to FIG. 1, which is a schematic flowchart describing adjustment and assignment of a regimen for delivery of at least one active agent to a user, based on feedback information from one or more other users, according to some embodiments of the present disclosure.

At block 102, in some embodiments, an inhaler is provided to at least one user with an initial dosage or regimen for inhalation of at least one active agent derived, for example, from raw plant material with which the user is provided for use with the inhaler. A user may use the inhaler for pharmaceutical purposes, for example, for treating a condition or a set of conditions, and/or for recreational purposes (such as selecting different sensations for different occasions and/or finding a preferred combination or blend of source material or active agents) and/or for any other purpose, including cognitive altering purposes.

The inhaler may be provided with a plurality of cartridges comprising the one or more plant materials. In some embodiments, the plant material includes plant material from one or more of: Cannabis sativa, Cannabis indica, Cannabis ruderalis, Acacia spp., Amanita muscaria, yage, Atropa belladonna, Areca catechu, Brugmansia spp., Brunfelsia latifolia, Desmanthus illinoensis, Banisteriopsis caapi, Trichocereus spp., Theobroma cacao, Capsicum spp., Cestrum spp., Erythroxylum coca, Solenostemon scutellarioides, Arundo donax, Coffea arabica, Datura spp., Desfontainia spp., Diplopterys cabrerana, Ephedra sinica, Claviceps purpurea, Paullinia cupana, Argyreia nervosa, Hyoscyamus niger, Tabernanthe iboga, Lagochilus inebriens, Justicia pectoralis, Sceletium tortuosum, Piper methysticum, Catha edulis, Mitragyna speciosa, Leonotis leonurus, Nymphaea spp., Nelumbo spp., Sophora secundiflora, Mucuna pruriens, Mandragora officinarum, Mimosa temuflora, Ipomoea violacea, Psilocybe spp., Panaeolus spp., Myristica fragrans, Turbina corymbosa, Passiflora incarnata, Lophophora williamsii, Phalaris spp., Duboisia hopwoodii, Papaver somniferum, Psychotria viridis, spp., Salvia divinorum, Combretum quadrangulare, Trichocereus pachanoi, Heimia salicifolia, Stipa robusta, Solandra spp., Hypericum perforatum, Peganum harmala, Tabernaemontana spp., Camellia sinensis, Nicotiana tabacum, Nicotiana rustica, Virola theidora, Voacanga africana, Lactuca virosa, Artemisia absinthium, Ilex paraguariensis, Anadenanthera spp., Corynanthe yohimbe, Calea zacatechichi, Coffea spp. (Rubiaceae), Sapindaceae spp., Camellia spp., Malvaceae spp., Aquifoliaceae spp., Hoodia spp. Chamomilla recutita, Passiflora incarnate, Camellia sinensis, Mentha piperita, Mentha spicata, Rubus idaeus, Eucalyptus globulus, Lavandula officinalis, Thymus vulgaris, Melissa officinalis, tobacco, aloe vera, angelica, anise, ayahuasca (Banisteriopsis caapi), barberry, black horehound, blue lotus, burdock, camomille/chamomile, caraway, cat's claw, clove, comfrey, corn silk, couch grass, damiana, damiana, dandelion, ephedra, eucalyptus, evening primrose, fennel, feverfew, fringe tree, garlic, ginger, ginkgo, ginseng, goldenrod, goldenseal, gotu kola, green tea, guarana, hawthorn, hops, horsetail, hyssop, kola nut, kratom, lavender, lemon balm, licorice, lion's tail (wild dagga), maca root, marshmallow, meadowsweet, milk thistle, motherwort, passion flower, peppermint, prickly poppy, purslane, raspberry leaf, red poppy, sage, saw palmetto, sida cordifolia, sinicuichi (mayan sun opener), spearmint, sweet flag, syrian rue (Pegamum harmala), thyme, turmeric, valerian, wild yam, wormwood, yarrow, yerba mate, and yohimbe and/or other plants and/or natural substances (e.g., mineral, bacterial, archea, fungal and/or animal material). Optionally, the plant material is provided with no additives or modification other than physical changes intended to reduce the plant material to small size particles. Additionally or alternatively, artificial materials and/or extracted and/or purified active agents are provided in addition to or instead of plant material. Additionally or alternatively, a placebo is provided in addition to or instead of one or more of the source materials.

It is noted that in some embodiments using plant material as a source material for at least one active agent may provide benefits that are associated with the natural complexity and diversity of plants. The same plant type might have different active agent combinations/proportions due to differences for example in strain, growth conditions, time of harvesting and post harvesting storage. Moreover, plants may sometimes display a so called “entourage effect”, whereby an active compound provides a different effect when provided alone than concomitantly with other agents that are co-extracted from the plant material. It is a particular feature of some aspects of the disclosure to benefit from such potential variability and/or diversity and/or complexity of active agents and active agent combinations delivered from such different plant materials and/or from each one, even if the precise composition of the material or extracted agents is unknown.

The inhaler may be provided with a plurality of cartridges comprising the one or more active agents. Optionally, each cartridge of a plurality of cartridges comprises a different substance and/or combination of substances. In some embodiments, a cartridge comprises a plurality of dose sections separately addressable for use. Optionally, each dose section of a plurality of dose section comprises a different substance and/or combination of substances. Examples include cartridges comprising different plant materials or plant material combinations and/or different ratios between different plant materials and/or plant materials having different active agent types, amounts, ratios and/or compositions.

The dosage regimen may include and/or indicate one or more of:
An amount of one or more active agents to be inhaled;
The timing of one or more of such inhalations;
A ratio between one or more active agents to be inhaled; and
A sequence of such inhalations.

In some embodiments, a relationship between amounts defined by the regimen and amounts actually released from a cartridge is maintained by the use of active agent extraction parameters defined for the cartridge. The cartridge optionally provides these parameters itself, and/or provides an indication (e.g., an identifier) that allows the parameters to be obtained from an external source such as a remote server. Optionally, the extraction parameters are defined per device type with which a cartridge may be used. Optionally, different parameters are provided for the cartridge, whereby each parameter or set of parameters allows the extraction of different amounts and/or combinations of one or more active substance(s) from the same cartridge. When the regimen is used with a particular cartridge (and optionally device type), the parameters of the regimen are satisfied by making suitable use of the extraction parameters to control extraction from the cartridge.

In some embodiments, for example when new source material and/or a new active agent are provided, a placebo is assigned to at least some of the users. The system may be configured perform this in a blinded to double blinded manner such as by control of disclosure of (e.g., barring access to) information to users and/or researchers and/or physicians. Such undisclosed information may include the specific regimen provided to a given user, to which user a placebo was or was not delivered and/or when such placebo was delivered to a user. The information may remain undisclosed indefinitely and/or may be provided at a time such as after the use of placebo was terminated and a regimen was stabilized, the termination of placebo use for a given patient and/or group of patients and/or specific circumstances mandating disclosure of the regimen (such as medical need for the data in treating the user for the same or any other condition).

In some embodiments, placebo administration (which in some embodiments comprises any administration of a substance which is substituted for a user-commanded and/or user-requested substance, dose and/or active agent; optionally without indicating the substitution to the user) is selected at least in part by operation of an active agent delivery device. In some embodiments, the device is provided with a plurality of doses, at least one of which comprises active agent, and at least one of which is a placebo for the active agent. Optionally, for example, the placebo gives a similar experience during administration (e.g., flavor, smoke), but does not comprise the active agent, comprises a different active agent (optionally less active), and/or comprises a weakened formulation of the active agent. The placebo is optionally selected by the device without informing the user of the selection. In some embodiments, the device itself selects (e.g., randomly, pseudo-randomly, according to a preset pattern, or otherwise) whether it will deliver a placebo or an active agent dose, delivers the selected dose, and reports the selection (e.g. via a network connection). In some embodiments, the choice of placebo dose or active agent dose to be delivered by the delivery device is made remotely, for example by or through a server, and the delivery device delivers the remotely selected dose. In some embodiments, the ratio of placebo doses to active agent doses is about equal. This is optionally the case, for example with a study requiring equivalent samples for either condition. In some embodiments, the ratio of placebo dose to active agent doses is relatively low (e.g., 1%, 5%, 10%, or another higher, lower or intermediate fraction of placebo). This is optionally used for occasional calibration purposes; e.g., to define what a “no delivery” profile looks like for a particular user, which optionally is used to help in distinguishing background reporting characteristics from reporting which is actually correlated with active agent delivery. In some embodiments, a placebo to active agent ratio is relatively high, for example 80%, 90%, 100%, or another lower or intermediate fraction of placebo). In some embodiments, a placebo dose is always delivered from a suitably configured delivery device, for example, as an inexpensive and/or effect-free training method for users, and/or for demonstration purposes. Optionally, several placebo doses are first given before an active agent dose in order to prepare a user for the actual dosing (e.g., during training) without wasting and/or mis-administering an actual dose. Optionally, a user who appears to be addicted, at risk for addiction, or having another reason requiring a lock-out from and/or reduction of active agent delivery is provided with cartridges comprising a significant proportion of placebo doses, which are optionally substituted for active agent doses with any suitable schedule (for example, randomly or pseudo-randomly interleaved with active agent doses, only if a user requests a second dose within too short a time, or every time).

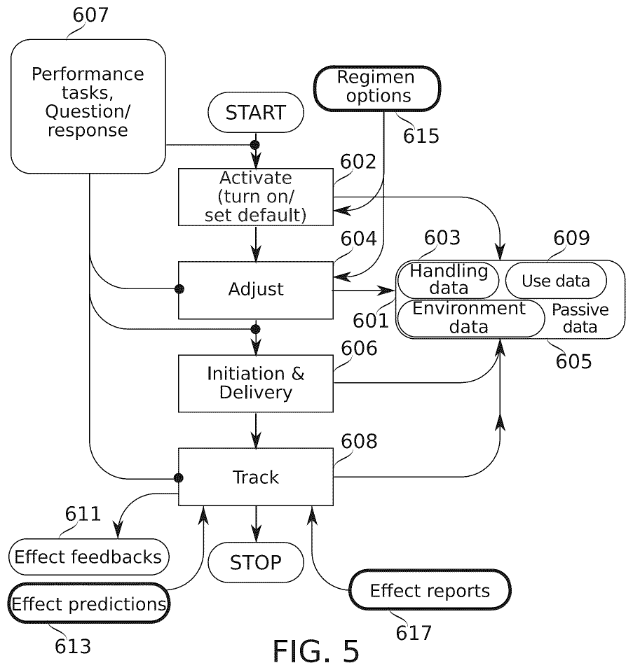

At block 104, in some embodiments, the user provides feedback regarding one or more effects of the regimen. This feedback may be provided at specific points in the regimen and/or at will. Feedback may be provided passively and/or actively (for example as described in relation to FIG. 5, herein). Optionally, the feedback is provided in real time, namely while the user is still influenced by the active agents.

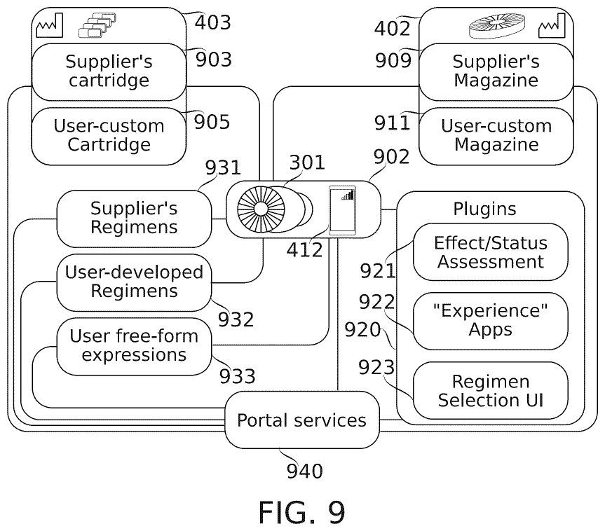

Feedback is optionally provided via an input system based on one or more inputs, for example: a user interface in mobile and/or wearable technologies and/or in the inhaler itself, environmental and human input (relatives, clinical personnel, and/or a caregiver; etc.), and/or one or more measuring devices (for example a blood pressure measuring device, a pulse measuring device, etc.). Optionally, feedback comprises selecting from multiple choices. Optionally or alternatively, a user can create a new choice (for example, as described in relation to user free-form expression 933 of FIG. 9, herein), optionally for other users to select and/or vote on. Optionally, feedback comprises selecting a degree or level of a specific response parameter, for example by moving a bar on a scale. In some embodiments, feedback is collected automatically and/or passively. Additionally or alternatively, feedback is provided voluntarily and/or semi-passively or actively (for example as defined and described in relation to FIG. 5, herein.

At block 106, in some embodiments, the user's own regimen (and/or that of one or more users) is adjusted according to the user's input (alone or in combination with inputs of other users). Optionally, any adjustment of a regimen is subject to approval by a medical practitioner and/or is restricted to imposed limitations (for example, in view of user safety). The adjusted regimen may be re-provided for assignment to the original at least one user at block 102. It is noted that the adjusted regimen is optionally provided for adjusted use of the same dose formulations already available to the user. Adjusted regimens are optionally determined and returned to members of a user population within minutes, hours, days, weeks and/or months of uses which provided input on which the adjusting is based. Optionally, the adjustment is provided no more than 6 months, no more than 2 months or no more than 1 month from receipt of the last user input on which the adjustment is based.

Additionally or alternatively, at block 108, in some embodiments, input from one or more users may be used to adjust the initial (and/or other) regimen provided to later users, essentially as described with respect to block 106. For example, an initial regimen for the first user (or group of users) comprises inhalation of 1 mg of tetrahydrocannabinol (THC) from cannabis, three times a day. Based on user input, the regimen of the example is adjusted by reducing the amount of THC per inhalation to 0.75 mg. Optionally, the adjusted regimen also includes increasing an amount of cannabidiol (CBD) per inhalation (e.g. by adjusting a strain of cannabis used or by combining a plurality of sections yielding a desired combination). Optionally, the adjusted regimen includes inhaling different CBD amounts with said THC dose at each inhalation. This new adjusted regimen may now be provided to new users that would use the inhaler for the same or a similar purpose. A potential advantage of dynamic adjustment is to continuously match (and optionally re-match) provided dose formulations to targeted effects. As use of a regimen becomes established among members of a user base, this process of matching may be understood as comprising a phase of initial refinement, wherein real-world experience with a regimen leads to improvements to reduce side-effects, increase targeted effects, better match adjustable regimen parameters to the profiles of individual users, define a desired sensory or psychoactive effect or combination etc.

The refinement is optionally performed as part of an early release and/or testing phase of a dose formulation. Optionally, regimen characteristics are fixed after a period of time, e.g., prior to general release to a larger population of users. Alternatively or additionally, regimen characteristics continue to be subject to dynamic adjustment for as long as a regimen (and/or its derivatives) is in use. In part, this continued adjustment potentially allows a regimen to be adapted to new understandings of how the regimen best functions within the lifestyles of individual users, incorporate new “tricks” (e.g., variations for adjusting to events like an acute illness) of discovered by users and/or by analysis of data collected from the user population. For example, after an initial refinement stage, a new magazine or set of cartridges is defined as the standard regimen for a given purpose. This new magazine or set of cartridges are then issued to users who are interested in the same treatment or purpose, while feedback is still collected from all or some of the users to further refine the regimen.

In some embodiments, changes to a regimen are introduced to maintain or restore established regimen effects. Optionally, the dynamic adjustment is used for self-standardization of lots. Potentially, this helps to compensate, e.g., for variability in botanical potency and/or active agent release characteristics which may arise due to differences in growing conditions, adjustments to manufacturing processes, differences in doses manufactured at different sites, etc. Optionally adjustment is assisted by reference being made to known properties of one or more of these conditions. For example, information related to growing conditions optionally comprises any one or more of breeding and/or plant variety data, growing technique, nutrition factors, lighting conditions and/or growth soil composition.

Dynamic adjustment potentially helps compensate for post-manufacturing variability of doses; for example, as manufactured dose preparations (e.g., packaged in cartridges) age through their normal shelf life. Optionally, dynamic regimen adjustment helps to compensate for regional and/or seasonal environmental effects, such as differences in heat/cold experienced by manufactured dose preparations during transport. In some embodiments, regimen adjustments are applied to assist users in adjusting to changes in their own responses to the regimen, for example, due to tolerance increases and/or changes in physical condition.

It should be noted that the adjustment is optionally performed in real time, at least in the sense that the timing of regimen determination is optionally decoupled from lags introduce by the physical supply chain comprising activities such as manufacturing and distribution. Crowd-sourced adjustments to regimens are optionally implemented without introducing a requirement to manufacture and/or distribute new formulations of dose preparations. A potential benefit is in that an adjustment may be carried out automatically by the device, without need for the user to change a routine (such as taking a half a pill instead of a pill), thereby potentially reducing non-compliance.

In some embodiments of the disclosure, during use by the second one or more users, additional feedback may be collected from the new users (represented by the connection between blocks 108 and 104), and optionally also from further uses by the original one or more users (represented by the connection between blocks 102 and 104). This may allow adjusting the regimen periodically and/or on an ongoing basis, for example to the benefit of all and/or later users.

In some embodiments of the disclosure, users are treated (at least partially) as members of a cohort which is optionally closed to new users at some point during regimen development (that is, new users would be treated as belonging to a separate cohort). In some embodiments, this helps to reduce disturbing effects which different properties of new users could have on existing regimen users (e.g., due to shifting criteria of user recruitment). Potentially, cohort designation allows a regimen to be reasonably adapted en mass to a user population which is simultaneously experiencing similar changes in their responses to the regimen, e.g., due to effects of tolerance and/or acclimatization. The “cohort” designation is optionally implemented only by operations of the input data analysis algorithms (AI algorithms, for example), making it transparent to the users themselves. Alternatively, cohort designation is known to users themselves, and/or to other users (suitably anonymized). Additionally or alternatively, crowd sourced research initiatives could group as “cohort”. Such designation is optionally used, for example, to reinforce a sense of identity and/or community among users, help users place reports and advice from other users in the perspective of their particular experiences (e.g., time of participation in the regimen), etc.

In some embodiments of the disclosure, feedback is collected using a user interface and sent, for example, via a wireless connection (e.g., cellular connection or Wi-Fi connection) or offline connection (e.g., USB connection or RFID) or a connection to another device (e.g., Bluetooth connection).

Optionally, each user can benefit initially from the group data (e.g., to receive the first regimen assigned to the user), while also applying an option to further adjust his own regimen based on his own needs and/or preferences. Optionally, the decision of a user to adjust their regimen, particularly if the adjustment persists, is treated as a type of feedback, e.g., asserting a preference for the new regimen over the old one. In some embodiments of the disclosure, voluntary adjustments by users of their own regimens are incorporated into the input data used in automatic regimen adjustment determination. In some embodiments, users are prompted before an adjustment is made to their regimen, allowing them to refuse such an adjustment if they are pleased with a current regimen setting.

Optionally, a regimen that was adjusted by using group information may be proposed to a user that was previously assigned use of an inhaler, to allow the experienced user to try the group result and possibly improve on personal adjustments.

In some embodiments, regimen adjustment comprises adjusting a “population regimen”, which is generally aimed at producing the same effects in diverse members of a user population, but comprising parameters which can be different per user, based on inputs which indicate differences in user effect responses and/or preferences by type. For example, a regimen optionally includes a parameter indicating sensitivity of individual users to a particular active agent (e.g., as determined from previous experience, user preferences, personal traits of the user, etc.) which governs differences in how that active agent is administered to each use. In some embodiments, adjustment of the population regimen comprises adjusting how individual user inputs are converted into actions of the population regimen itself—e.g., how in timing and/or delivery amounts are changed along the scale from most sensitive to least sensitive for any given protocol.

In some embodiments of the disclosure, regimen adjustment at block 106 and/or data collection at block 104 take advantage of artificial intelligence (AI) capable of analyzing high quantities of information at high speeds, potentially allowing quick and efficient adjustment of regimens, and/or detection of trends (e.g., small but significant trends distributed among several interacting parameters) which human review might miss.

For example: in some embodiments, an artificial intelligence (AI) driven computer system is provided. The AI system may be driven by machine learning processes such as neural nets; e.g. deep learning algorithms, and/or evolutionary computation. Based on the collected data, the AI may adjust a regimen or recommend an adjusted regimen. AI may combine information relating to a specific patient (e.g. personal traits such as age, gender, weight, background and medical history, occupation, genetic information, etc.) as well as other circumstances (time of day, activity during use, and more). Combining the data may allow the device to tailor an initial regimen to each patient or each group of patients-either as a “population regimen” accepting user parameters making it suitable to apply to a wide variety of user types, or as a regimen (optionally derived from some parent regimen) specialized for a certain user type. For example: a young thin active patient may require or desire less sedation and pain reduction than an elderly overweight bedridden patient, who potentially prefers the opposite. Machine learning may be taken to mean a subfield of computer science that evolved from the study of pattern recognition and computational learning theory in artificial intelligence. Machine learning may be used to explore the study and construction of algorithms that can learn from and make predictions on data.

Evolutionary algorithms (EA) are a subset of evolutionary computation, a generic population-based metaheuristic optimization algorithm. An EA uses mechanisms inspired by biological evolution, such as reproduction, mutation, recombination, and selection.

Optionally, the AI may initiate polling and medical inquiries with the users and inquire on the specific condition of the patient at a specific time based on the regimen pulse provided.

In some embodiments of the disclosure, the AI is provided in the form of circuitry, programming, and/or any combination of the two. In some embodiments, systems configured for AI-based learning yield results in the form of trained networks, selected weighting parameters, etc., which are provided in any suitable form for use with systems that may be non-learning systems. For example, an AI implementation in some embodiments collects inputs and “learns” associations for adjusting regimens based on particular inputs. The learned associations are converted into a form (for example, a software module and/or updated database; either being optionally for a network-connected portal site and/or for use on a user-operated device) which is made available to users, allowing users to take advantage of AI-produced results without necessarily requiring user-level direct access to AI-based learning systems.

Some of the methods described herein are generally designed only for use by a computer, and may not be feasible or practical for performing purely manually, by a human expert. A human expert who wanted to manually perform similar tasks, such as searching for patterns in large amounts of data, might be expected to use completely different methods, e.g., making use of expert knowledge and/or the pattern recognition capabilities of the human brain, which would be vastly more efficient than manually going through the steps of the methods described herein.

A potential problem of basing algorithmic (e.g., AI-generated) regimen adjustment on user inputs is vulnerability to a certain type of “trolling”, wherein a subgroup of one or more users attempts to deliberately train the AI to output and/or allow regimen recommendations having effects which are outside of intended parameters. For example, users (with or without legitimate system access) may deliberately under-report effects of an active agent in an attempt to elicit an increase in delivered amounts. In principle the reverse might also be attempted. Optionally, this may be defended against by placing hard limits on how much (or even if) a regimen is allowed to “evolve” without oversight from a human operator to validate the reasonableness of modified results. In some embodiments, inputs from users who appear to systematically provide distorted inputs are excluded as inputs to the AI algorithm. Optionally, AI itself is used to detect such users. For example, trusted users (and/or developers, and/or other professionals) devise plausible input-distortion attacks on the system. The AI is optionally trained (e.g., based on a suitable training set comprising simulated or actual attacks) to recognize patterns inherent in such attacks as signs that the input source should not be trusted. Additionally or alternatively, “hard rules” are set that block suspicious input patterns, blacklist sources already considered untrustworthy, and/or flag suspicious input for human investigation.

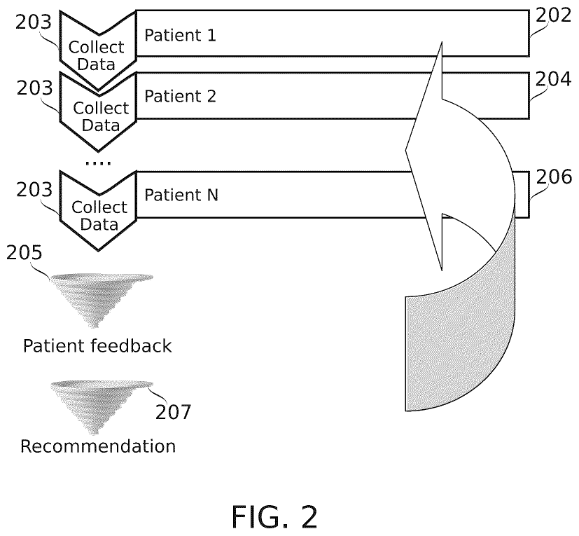

Reference is now made to FIG. 2, which schematically illustrates an example of data collection from a plurality of patients undertaking an inhaler-delivered treatment regimen for a condition, followed by output of recommended adjustments to treatment regimens, according to some embodiments of the present disclosure. The example of FIG. 2 is described with respect to treatment of parkinsonism disorders with high THC cannabis. It should be understood that the same operations may be applied in some embodiments of the disclosure, changed as necessary, for treatments of other conditions, based on the use of the same and/or other active agents. Parkinsonism disorders include a wide range of different primary causes and associated features. Patients can differ significantly from one another, not only in the underlying type of the disease, but also in disease stage, and/or general patient characteristics such as age, weight, sex, etc. In view of this complexity, there is a potential benefit for personalization of treatment systems that can associate input data specifics to suggested treatment regimens and/or suggested adjustments to treatment regimens.

In this example, each patient 202, 204, 206 is provided with an inhaler device for treating his disorder. As schematically shown, a first patient's 202 input may affect a second patient's 202 initial regimen and so forth, with later patients potentially benefiting from the experience and feedback provided by earlier users. Each patient 202, 204, 206 may provide feedback 205. This feedback 205 is optionally collected (at block 203) and/or analyzed by an AI implementation. Feedback 205 optionally includes input regarding the patient's symptom(s), which in the case of THC treatment of parkinsonism optionally comprise at least one measure of tremor (e.g., a stand-alone measure of tremor; a degree of tremor reduction, if any; or degree of tremor compared with prior input, prior trend, and/or a baseline degree of tremor). Additional input may relate to side effects (e.g. sedation). Optionally, the patient provides input regarding a degree to sedation. Optionally, the patient provides input regarding the desirability of the side effect (e.g. sedation may be desired at night and undesired during daytime). Optionally, additional data is used (collected initially and/or updated at any suitable interval) which characterize, for example, associated features of the disease and/or patient history.

The adjusting of the regimen (and/or adjustment recommendation 207) may include, for example:
Reducing/increasing the amount of one or more active agents in the next dose or at specific doses (based on other regimen properties, such as time of day and/or doses taken before and/or after a dose). This optionally includes removing and/or adding active agents. In some embodiments this may include changing the extraction parameters without changing a source material, thereby changing the amount of one or more active agents extracted therefrom.
Adjusting a composition or ratio of active agents in a dose (e.g. by replacing one amount of cannabis with another having a higher CBD:THC ratio)
Changing regimen features; including, for example, reducing the amount of active agents inhaled in early morning, while increasing the amount inhaled in the evening.
Administrating a long treatment plan regimen with a desired end result goal (e.g. weight loss, detoxification, withdrawal etc.)

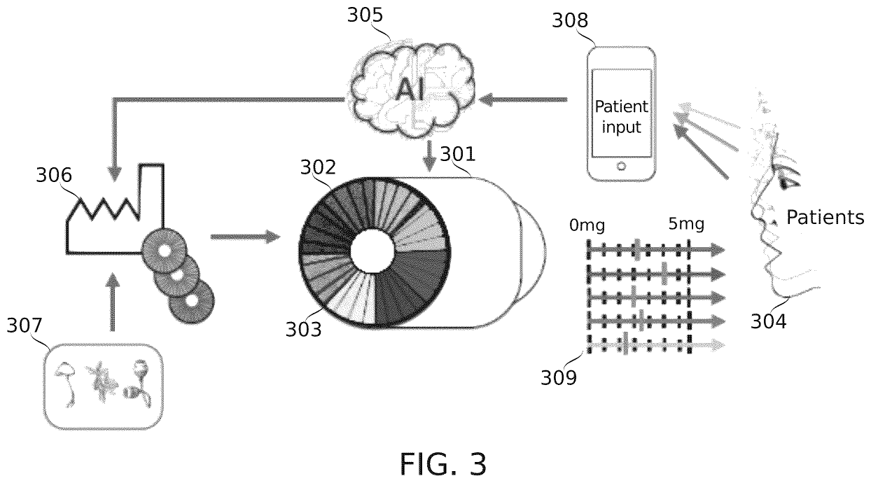

Reference is now made to FIG. 3, which schematically represents use of an autonomous, continuous, multi active agent, selective dose-discovery, -delivery, -adjustment and/or -optimization system, according to some embodiments of the present disclosure.

A metered dose active agents delivery device, optionally an inhaler 301, may be used with one or more replaceable magazines 302 which hold multiple cartridges 303, of various active agents and/or active agent amounts and/or active agents compositions (represented by cartridge zones 304 having different shadings) which optionally comprise raw and/or extracted botanical material and/or synthetic material 307 which was manufactured by a manufacturer 306 into predetermined doses (e.g. weighed and/or having a known amount or concentration of one or more active agents). Optionally, in addition to or instead of magazine 302, one or more cartridges 303 are provided separately, each having a plurality of dose sections. In such event, the cartridges or sections within a cartridge may differ in the active agents and/or active agent amounts and/or active agent compositions, which optionally comprise raw and/or extracted botanical material and/or synthetic material. Optionally, the magazines and/or cartridges are tagged (e.g. by RFID or other identifiable marking), which are used by the system to verify compatibility between the source material or active agent combination in the magazines and/or cartridges and a given regimen, and/or to provide a regimen that is applicable for the user's condition and may be provided with the provide source material or active agent combination. Optionally, based on this information the AI may instruct the user to obtain or provide the user with a new magazine or cartridge to support desired regimen that cannot be applied with the available material.

In some embodiments, inhaler 301 provides the option to select and load or directly use specific cartridges from among the multiple cartridges 303 in the magazine 302 and/or specific sections within a cartridge 303, and deliver a range of active agents from said cartridges. In the case of botanical materials, the available range of deliverable active agents may also include variation according to differences in the process by which the active agents are evaporated or otherwise extracted from the plant material.

In some embodiments of the disclosure, a plurality of different botanicals are provided with an inhaler 301, wherein at least some of the botanicals are to be taken in some combination—concurrently and/or sequentially—defined by the treatment regimen. The botanicals may vary, for example by strain, amount and/or composition of active agents, preparation of the botanical, amount of botanical and/or the kind of plant.

In some embodiments the inhaler is loaded with multiple botanicals, for example two or more of: Cannabis strain with relatively high THC (e.g. Bedrocan), Cannabis strain with relatively high CBD (e.g. Bedrolite), Opium, Amanita muscaria, Psilocybe cubensis, Valerian, Khatt, Sceletium tortuosum, Tobacco and a placebo.

The cartridge's contents are delivered to a plurality of patients 304 (e.g. 50 patients or more or even 100 patients or more) having the same or similar condition (e.g. a neuronal and/or psychiatric disease, parkinsonism disorder, multiple sclerosis, autism, obsessive compulsive disorder, etc.). Additionally or alternatively, the disease or condition is assessed upon usage, based on treatment effects on symptoms. Optionally, the patients receive same-configured cartridges 303, comprising any one or more of botanicals 307 (selected, for example, from botanicals 1-8 from the foregoing list), and the inhaler 301. Optionally, cartridge 303 selection is customized, for example, based on past results of treating the patient and/or the patient's medical history.

The patients 304 load the cartridge 303 into the inhaler 301, or receive it preloaded. Optionally the inhaler 301 is preset with a regimen. Additionally or alternatively, the inhaler communicates with a database (e.g. via the internet to the AI system 305, and/or to a system configured to provide results from the AI system) to receive a regimen and/or adjust a pre-set regimen.

In some embodiments of the disclosure, the AI system 305 transmits a specific regimen to each inhaler 301 based on the available one or more botanicals 307 in the magazine 302 or cartridge 303, and/or based on an initial regimen (e.g. defined by a physician). Optionally, the regimen provided is maintained within a set safety range of dosing for each active agent and/or for active agents combinations. The regimen may comprise or consist of any sequence of active agents and selective dosing the inhaler is capable of delivering. Optionally, one regimen is provided to everyone. Optionally, regimens are varied, for example based on the initial conditions, requests and/or preferences of each patient.

Optionally, a range of regimen variants is provided to the patient population 304 (one variant per patient or per group of patients). This may have the potential advantage of allowing more rapid accumulation of feedback data covering different regimens and/or initial conditions, although it can also risk increasing levels of statistical noise in the results obtained for specific regimens. For example, each of a plurality of initial regimens, expected to be beneficial, is assigned to a subset of users. Optionally each user uses a single initial regimen or one or more of the users use a plurality of regimens in sequence, thereby providing comparative data.

In some embodiments, each user of a plurality of users (e.g. patients) receives and is administered a plurality of different regimen variants (for example, 2, 3, 4, 5 or more regimen variants) over a period of time. Regimen variation optionally comprises, for example, differences in active substance composition. Optionally, the differences in composition comprise different plant material compositions, different plant species, different amounts of extracted active agent(s) and/or different plant strains and/or varieties. In some embodiments, the regimen variants are administered (in any suitable order), for example, over the course of about a week, about two weeks, about a month, about six months, or for another suitable period. This method of administering regimens provides a potential advantage, particularly for regimen testing, since feedbacks are optionally controlled inherently for inter-user variability.

Initial regimens are delivered to patients, and feedback 308 is collected. The AI system takes into account the feedback (along with conditions that produced it) and provides adjusted regimens in response. The initial regimen may relate to a single delivery event or to a sequence of delivery events spanning a period of, for example, an hour, a day, a week, a month, 6 months, a year or any longer, shorter or intermediate period.

The adjusted regimen(s) 309 is/are targeted to provide an improved therapeutic effect to said population compared with initial regimen(s). The adjusted regimen(s) may select from amongst the initial regimens available and/or produce a new regimen based on input from the plurality of initial regimens. In some embodiments, the adjusted regimen(s) comprise a selected regimen from among a plurality of regimens originally administered.

In defining a regimen, the AI system 305 may interchange source materials (e.g. botanicals), alter dosing sequences, dosing levels, active agent combinations and/or ratios and/or any other parameter controlled by the system. The system optionally enacts placebo doses and/or regimens for any statistical and validation processes which may be warranted. Providing a placebo may be performed in a blinded and/or double blinded manner.

Optionally, basis for the adjustment comprises assumed covariance of dosing amount/periods with effects and/or side-effects (e.g., more active agent, and/or active agent delivered more often, leads to greater effects). Optionally, particular response curves for parameters of the regimen (e.g., how much more active agent yields how much more response and/or when increasing the amount is no longer beneficial) are learned from relative responses to different regimens. When machine learning is used, such response curves are potentially discovered in the data as part of the learning procedure, implicitly or explicitly. Optionally, learning of response-curve like information derives from systematically different reactions to similar regimens delivered to patients who provide different background conditions. The different background conditions are optionally available, for example, based on provided assessments of patient history, disease progression, disease subtype, associated conditions, etc. There may be prior assumptions used as to how different parameters affect responses, for example, higher weight may be assumed in a machine learning model to have some kind of diluting effect on treatment regimen results; the magnitude of the particular hypothesized effect, if any, is learned from the feedback data.

Optionally, one or more cartridges 303 are replaced in adjusted regimens with another set of active agents to be incorporated in the inhaler, thus creating new multi-cartridge, multi-active agent combinations, as the AI results determine. Optionally, the new cartridges 303 include one or more cartridges or source materials (e.g. botanicals) previously not provided in the original magazine 302.

Optionally, input is continuously or periodically collected from later and/or continuing users to further adjust the regimen. Optionally, as data is collected, users may be divided to sub-groups based on population statistics that are found to correlate with different regimen requirements or preferences. This process of division can help with increasing the applicability of machine learning results to particular sub-populations, while potentially decreasing input noise from unrelated populations. For example, it may be that women or children may benefit from a significantly different regimen than men or elderly patients suffering from the same condition. Relevant parameters may include age, gender, race, weight, activity, lifestyle, other medications and/or conditions, medical history, etc.). In some embodiments, populations of disparate users are kept together within one paradigm (that is, one paradigm considered as the target of adjustment), and the paradigm itself is adjusted for application in individual cases by applying suitable user type-dependent values to parameters of the paradigm.

Optionally, patients have access to modify regimens and may elect to manually configure regimens. In some embodiments, such elected adjustments and/or their reported effects are fed back into the AI; e.g., reported effects on therapeutic results being positive or negative or neutral. Optionally, system recording of the change as such (and/or a change back, should the new regimen be rejected by the user) is treated by the AI as a result reflecting on the effects of one or both of the old and the new regimens.

The system is optionally utilized for non-medical purposes, such as cognitive altering or manipulation to achieve a desired cognitive effect (e.g., enhanced alertness, enhanced concentration, lower fatigue, increased stamina and/or reduced fear and/or anxiety) and/or recreational desires. User populations in such event may be selected based on desired effects.

In some embodiments, the system may define a regimen for recreational use or cognitive altering purposes. For example, the system provides a magazine (and/or cartridges) having different source material (for example tobacco blends, cannabis blends and/or combinations thereof; optionally with one or more added flavor and/or scent agents). The regimen may define at least one or more one or more of the timing, order, amount, type and composition of source material, dose and frequency of delivery (e.g. by inhalation). Based on input from a plurality of users, essentially as described herein, the system may adjust the regimen to maximize user satisfaction and/or to obtain a desired cognitive effect. Optionally, the system does not provide or impose a regimen, but rather allows the user to deliver the active compound(s) at will and in any desired combination (possibly with some restriction such as limiting maximal doses of specific active agents). In such embodiments, the system may provide suggestions to a user (e.g. a blend, a combination, a sequence, timing, a frequency, an amount of active agent or even a full regimen). Among the potential outcomes of such a system are: (A) a regimen for a period of hours, a day, a week or longer. Such regimens may provide different sensations based on time of day (e.g. morning being different than evening) or activity (after lunch being different than while driving to work); (B) regimens adapted to specific populations and/or activities; and (C) a sequence of deliveries that defines a single pleasurable delivery event. In this case, the sequence may be designed similarly to smoking a cigarette, with the system tailoring a variation of sensation during smoking. Magazines and/or cartridges may be produced and marketed as SKUs (e.g. an electronic cigarette or inhaler) according to the results.

In some embodiments, patients and/or users optionally access an online portal in which they interact directly with the order and delivery of their requested cartridges. The portal may also support other uses, for example, reporting of experiences, reviewing of other user's experiences, browsing and/or selection of available regimens, etc. Physicians and researchers optionally enter the same portal (for example, at a different permitted access level) to monitor and/or control treatment processes.

### Examples of User Interface, Sensing, and Communication Devices

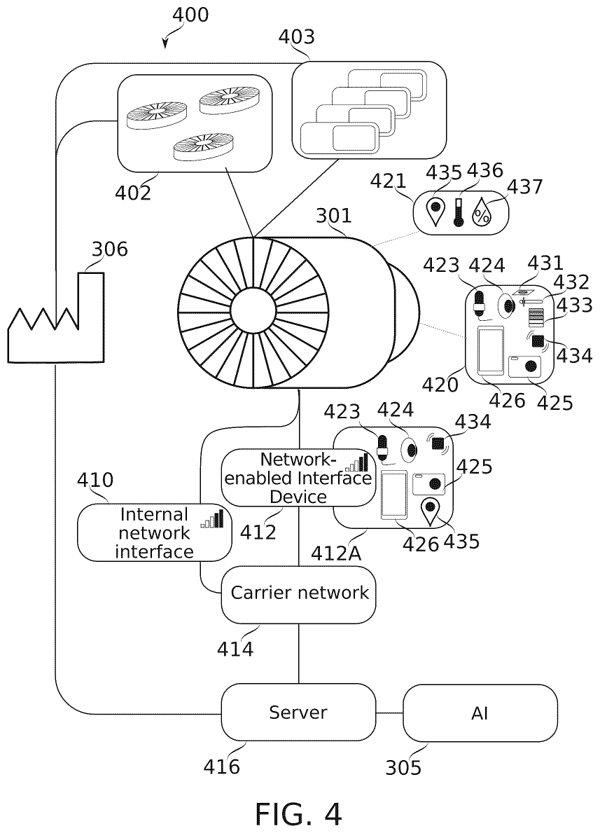

Reference is now made to FIG. 4, which schematically represents a system 400 for adaptive operation of an inhaler 301 based on inputs from a plurality of users, according to some embodiments of the present disclosure.

In some embodiments of the disclosure, inhaler 301 is provided with at least one device of a user interface 420, configured for providing feedback from and/or information indications to a user of the inhaler 301. The user interface device 420 may be an integral part of inhaler device 301 (such as within a single device housing) or otherwise associated therewith. It should be understood that system 400 comprise a plurality of inhalers 301, for example, one inhaler 301 for each of at least 10 users, 100 users, 1000 users, 10000 users, or at least another larger, smaller, and/or intermediate number of inhalers 301. It should also be understood that an apparatus used to deliver active agent optionally comprises a delivery device for active agents other than an inhaler; for example, a suitably configured injector or other transdermal delivery mechanism, and/or a pill dispenser.

In some embodiments, the user interface 420 optionally includes any one or more of the following input and/or output devices: microphone 423, speaker 424, camera 425, touch screen sensor and/or display 426, motion sensor 434, indicator display 433, button or keyboard 431, and/or slider 432.

Additionally or alternatively, in some embodiments, one or more sensing devices 421 are provided. Optionally, sensing devices 421 comprise a medical measuring device, for example, a blood pressure measuring device and/or a pulse measuring device. Other examples of optional devices include, for example, location sensor 435, temperature sensor 436, and/or humidity sensor 437. Optionally, sensing 20) devices 421 include any sensors which collect data as part of the operation of inhaler 301, for example, internal temperature and/or flow sensors and/or pressure sensors, and/or humidity sensors and/or location sensor (e.g. GPS). In some embodiments, sensing devices 421 also includes means to accumulate and report actual sequences, timings, and/or other settings used with an inhaler 301 during operation of the device. Optionally, data collected by sensing devices 421 is provided by wired or wireless communication to the network. Additionally or alternatively, data collected by sensing devices 421 is provided to a user or a care giver who inputs it via a user interface 420.

In some embodiments of the disclosure, data acquired by and/or transmitted to user interface 420 and/or sensing devices 421 is communicated between inhaler 301 and a server 416 (e.g., via a carrier network 414) using an internal network interface 410 of the inhaler 301. Internal network interface 410 may implement, for example, a Wi-Fi or other IEEE 802.x protocol (preferably but not exclusively a wireless protocol), a cellular communications protocol (e.g., GSM, UMTS, CDMA2000, and/or LTE) and/or another wireless (preferably, but optionally wired) network protocol.

Additionally or alternatively, communication is via local point-to-point communication established (e.g., via a suitable wired or preferably wireless communication protocol such as Bluetooth or a near field communication protocol) with a network-enabled (that is, comprising a network interface device) and network-connected (as available) interface device 412. Optionally, the point-to-point communication device used by the inhaler 301 is considered to incorporate the inhaler into the network that connected interface device 412 communicates with, so that any such communication device is optionally considered to be a type of network interface. Interface device 412 may be, for example, a telephone, wearable, and/or tablet computing device, a laptop computer, or any other suitable device operable to serve as part of a user interface for use with inhaler 301. Optionally, any of the sensing devices and/or user interface devices (for example, and not exclusively, devices 423, 424, 425, 426, 431, 432, 433, 434, and 435) described in relation to user interface 420 and/or sensing devices 421 (and/or other devices suitable for sensing and/or interfacing with a user) may be provided as part of the sensing/interface capabilities 412A of interface device 412. Such interface/sensing capabilities 412A are optionally provided instead of and/or in addition to devices provided integrally with inhaler 301.

Optionally, interface device 412 includes implementation of a software application (“app”) through which inputs from and/or outputs to user interface 412A are mediated. Additionally or alternatively, a software driver and/or application active on interface device 412 operates one or more of the input and/or output devices of user interface 420, e.g., via use of a wireless communication protocol. Additionally or alternatively, inhaler 301 itself comprises a controller suitably programmed to operate user interface 420, including operations based on communication to server 416 over the internal network interface 410 of inhaler 301, and/or mediated through a data connection to interface device 412.

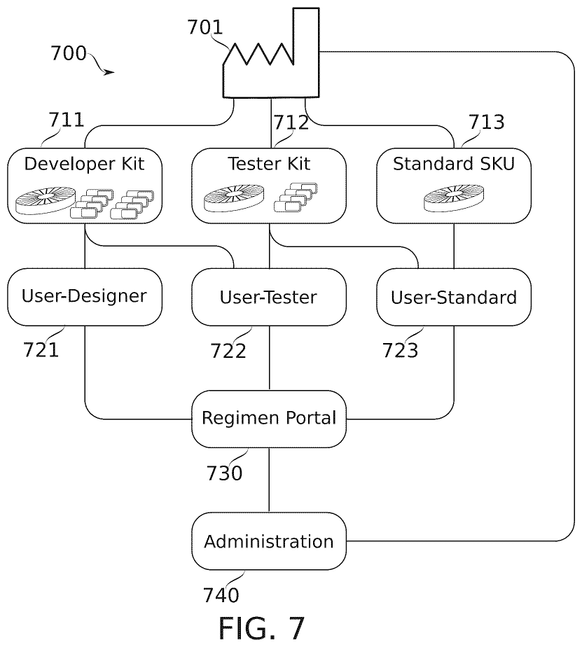

In some embodiments, there is also a form of “communication” which is mediated through the configurations available from a supplying manufacturer 306 of magazines 402 and/or of individual cartridges 403 which may be arranged in magazines 402, and/or may be used in the inhaler 301 directly. In some embodiments of the disclosure, new types of cartridges 403 and/or new load-outs of cartridges 403 provided, optionally in magazines 402, are made available for use with inhaler 301, based on usage patterns detected, e.g., by AI 305 based on input data accumulated by server 416. Optionally, new cartridge and/or load-outs are suggested and/or designed based on explicit user feedback. Optional arrangements for such a dynamic supply system are discussed, for example, in relation to FIG. 7 herein.

### Patient Feedbacks to the System

Reference is now made to FIG. 5, which schematically represents modes of user feedback data collection and modes of providing to a user inhaler use guidance and/or indications, according to some embodiments of the present disclosure.

### Passive Feedback Data Collection

In some embodiments of the disclosure (as schematically depicted in FIG. 4), user interface 420, sensors 421 and/or sensing/interface capabilities 412A are particularly configured for collection of feedback data related to use of the inhaler 301. In some embodiments, feedback data collection is by modes of operation that integrate with the overall user experience of inhaler use, examples of which are described hereinbelow.

In some embodiments, feedback data are collected such that reporting use data from an inhaler is implemented substantially as a function of the use of the inhaler for its primary function of delivering active agents. Herein, this type of collection is referred to as “passive” data collection 601 (“passive” as in requiring no additional actions; i.e., the user would perform essentially the same actions with or without the reporting function). Potential advantages of passive data collection include reduced user burden, increased reporting “compliance”, and/or reduced opportunity for injection of user error or bias into reported data. Another potential benefit is that at least some passive data collection 610 may be performed regardless of user ability to provide it. This may have the benefit of collecting data from users with diminished capacity (e.g. sick and/or very young or old or otherwise impaired users). Another potential benefit is that at least some passive data collection 610 may be performed regardless of user desire to provide it, thereby potentially detecting abuse and/or misuse (including deliberate and unintentional misuse).

In some embodiments, frequency and/or timing of use of the device itself and/or frequency and/or timing of providing feedback to the system by the user provides indicative information; for example, indicative of relative regimen success or failure. Potentially, reduced use of a regimen by a user and/or ceasing of a chain of feedback responses indicates “success” of the regimen (e.g., it is no longer needed as often to treat symptoms). However, reduced use and/or reduced reporting may also indicate “failure” of the regimen (e.g., a user gives up on it due to intolerable side effects, lack of desired effect, or another reason). Similarly, increased use or reporting potentially can be either a positive or negative indication (for example: negatively, increased use may indicate user desperation and/or addiction causing substance abuse; or positively, user satisfaction). In some embodiments, a system 400 distinguishes between such possibilities by the association of use frequency and/or timing with other input data indications; for example, symptom trends and/or satisfied/dissatisfied user feedback or cessation of feedback information following a chain of complaints as opposed to a chain of positive reports. Such relationships are optionally found by human supervised inspection of data, and/or learned by a suitably configured machine learning implementation. Usage timing can also be indicative, for example, relating to time of day, and/or clustering of uses. Where such patterns are associated, for at least some users, with other indications which are positive or negative, human supervised inspection or machine learning optionally help to identify whether the usage pattern itself comprises positive or negative feedback. Optionally, frequency and/or timing-related usage data, suitably interpreted as a positive or negative indication, are used in the development, refinement, and/or selection of regimens. Optionally, another action, for example, reporting to a physician and/or changing of inquiry rates to a user is performed on the basis of usage frequency and/or timing.

In some embodiments, collection of passive data 601 comprises collection of data related to handling (handling data 603) and/or data related to operation (use data 609) of the device. In the case of treatment of parkinsonism, for example, a motion detection device 434 (provided as part of either one or both of user interfaces 420, 412A) is optionally used to collect data indicative of a level of tremor evidenced during normal handling of inhaler 301 and/or interface device 412. In some embodiments, passive data collection also includes sensor and/or operational data (use data 609) 30 obtained during operation of inhaler 301 to deliver active agent; e.g., data related to airflow, temperature, valve operations, etc. Sensor and/or operational data potentially provide information indicating aspects of delivery of active agent such as successful/potentially unsuccessful delivery of the active agent.

In some embodiments, passive data collection optionally includes collection of environmental context data (environment data 605); for example, one or more of time, geographical location, device orientation, temperature, and/or humidity data. Such passive data potentially indicates aspects of the conditions of use of the device (time of day, indoor/outdoor use, climate, use while reclining or sitting/standing, etc.). Environmental data is not necessarily acquired by direct sensing on the inhaler 301 or interface device 412. Optionally, climate-type data (temperature and/or humidity, for example) are at least partially inferred from generally available information such as weather reports, indexed to location. Optionally, location data is determined, for example, by analysis of network network paths, IP addresses etc. By “location data” is not necessarily meant “individual location” data; in some embodiments, location data is available and/or aggregated only at a regional level. Use of region location data optionally helps to identify usage trends that have a local and/or regional component to their distribution and/or spread.

In some embodiments, sensing of environment data 605 comprises data (e.g., temperature and/or humidity data) read from a history accumulated by one or more sensing and/or recording devices originally packaged along with the plant or other material carrying the active agent to be delivered. The data optionally comprise a time-sequence of recorded values. Optionally, the data comprise recorded minimum and/or maximum values, recorded, e.g., by a single use mechanical and/or phase change type temperature sensor. Upon preparation for use with the inhaler, stored environment data is optionally read, and provided to server. A potential advantage of this is to provide a basis for correlating user experiences with the possibility of shipping and/or storage-related loss of active agent efficacy.

Environment data 605 optionally comprise data such as light (light level for example) and/or sound. In some embodiments, a microphone 423 senses ambient noises around the time of active agent delivery. Optionally, microphone sensing includes detection of noises indicative of respiratory activity; for example, respiratory activity that may have affected substance delivery, such as coughing, sneezing, and/or simple the time of exhalation after substance delivery.

In some embodiments, any collection of user data, and collection of passive data in particular, may be viewed by some users as intrusive; particularly where the data may tend to reveal something about a user's identity, habits, medical condition, etc. Applicable privacy laws also potentially govern the degree to which data (particularly medical data) may be collected and/or used without appropriate consent and/or anonymization. Optionally, data which is potentially subject to one of these or another privacy concern is collected on an opt-in and/or opt-out basis, and/or in an anonymous fashion (optionally including handling such as at least partial unbundling and/or binning of data streams, to resist and/or prevent de-anonymization).

### Semi-Passive Feedback Data Collection

In some embodiments, user interface data is collected as part of inhaler operation by tying the providing of user input to events that activate 602, adjust 604, and/or initiate 606 use of the inhaler 301. Acquisition of such data may be implemented in a “semi-passive” manner—that is, based on inputs which involve deliberate user interaction, but wherein the user interaction is not (at least from the user's perspective) specifically directed to providing the input which is obtained.

For example, in some embodiments, steps which for which some type of user interaction is anyway needed are implemented as more detailed (or otherwise distinguished) tasks than operation actually requires, so that these interactions also serve as data collection opportunities. Examples of that include small puzzles, timing tasks, and the like.

In some embodiments, activating (“arming”), adjusting, and/or initiating (“triggering”) an inhalation requires a user to perform an operation that the system 400 treats as a source of input data 607. In some embodiments, for example, a user is requested to press a button (displayed on a screen 426 and/or provided as a separate hardware button 431) upon delivery of a stimulus. The stimulus optionally comprises, for example, a tone (e.g., via speaker 424) visual indication (e.g., via a screen 426 and/or indicator display 433), and/or tactile indication (e.g., activation of a shaking device, for example, of a cell phone). In some embodiments, the input used as a part of input data 607 comprises a characteristic of the user response, for example, reaction time, sensitivity to a stimulus threshold, and/or accuracy of the response. In some embodiments, the input is treated as the result of a performance task; for example, to set a context and/or baseline for use in assessment of other data to be acquired.

Optionally, the input data 607 are interpreted as a response to a question, for example, a response to a yes-no and/or multiple choice question which the stimulus actually or is understood to “ask”. Questions may be, for example, related to configuration of the device, and/or to the status of the patient. In some embodiments, the stimulus is literally a question (displayed by screen 426 and/or automatically voiced through a speaker 424). Optionally, the input provided in response is literally a spoken answer to the question; optionally recorded by a microphone 423, and interpreted locally (e.g., by software on the inhaler 301 and/or interface device 412 itself), and/or remotely by server 416 and/or one or more server-side computerized systems (for example, an AI system) with which server 416 is in communication. In some embodiments, voice data (from commands, answers, etc.) are analyzed for content other than verbal content; for example, analyzed for sound features associated with stress, drowsiness, exhaustion, etc.

Optionally, operation of the inhaler for active agent delivery is gated to successful completion of such a task/answering of a question. Additionally or alternatively, failure to perform the task/answer the question (a user may legitimately be unable and/or unwilling to do it under some circumstances, and/or the task may not be relevant to each use) is simply scored as “no data”, either at the time, or upon later analysis.

### Active Feedback Data Collection

In some embodiments of the disclosure, feedback data comprise inputs given during a tracking period 608 after delivery of an active agent by a user indicating effects of the delivered active agent (effect feedbacks 611). In some embodiments, these inputs are given “actively”, that is, as inputs which the user explicitly provides, e.g., for purposes of reporting, complaining, confirming and/or simply sharing. In some embodiments the inputs are provided or required or requested before delivery of an active agent, relating to the previous delivery of an active agent.

Optionally, feedback relates to other aspects related to the experience of inhaler use; for example, opinion as to the value of inherent and/or added scents and/or flavors. Optionally these additions are made for increasing enjoyment or reducing discomfort from an inherent scent and/or flavor. Optionally, such additions are deliberately unpleasant, e.g., provided to a user who is attempting to manage a habit by adding negative reinforcement to at least some uses of the inhaler. In such events the system may be used to fine tune the degree of discomfort so as to best manage the habit without deterring the user from using the device. Optionally, such feedback is used to control the discomfort and treatment continues, so as to respond to habituation and acclimation.

Such feedbacks are optionally be passive or semi-passive, substantially as described already; for example, as the inhaler continues to be handled. Optionally, the feedbacks are active. The tracking period is potentially a fruitful period for obtaining willing active feedback, since a user may be focused on awaiting the effects for which the active agent was administered.

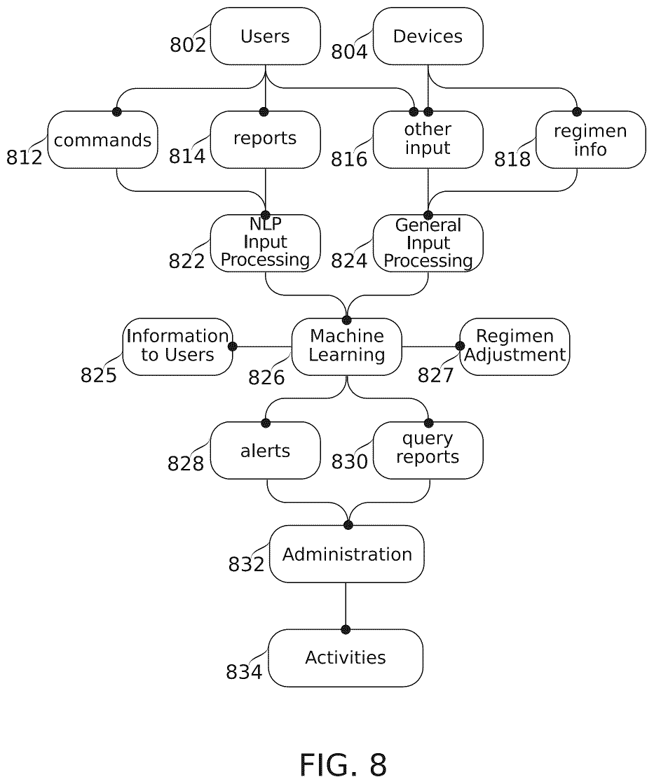

Active input is optionally delivered continuously and/or periodically; as prompted and/or as desired to indicate “greater” and/or “lesser” effect. Optionally, such input is delivered by pressing a button (or one of a plurality of buttons), through manipulation of a slider, through a software interface displayed on a touch screen device, or by another other suitable method. Optionally, multiple effects (e.g., at least one targeted effect and/or one or more side effects) are judged by a user in such a fashion. In some embodiments, active feedback comprises verbal input provided by a user and/or assistant to the user, for example as described in relation to the commands 812 and reports 814 described herein in relation FIG. 8. Another form of active feedback includes user usage patterns. In such embodiments, a user is provided with a selection of source materials and/or active substances and/or ranges of dose amounts and is permitted to use at least some of them freely. In such cases, the choices made by the user (even without any regimen guidance) may be used to define and/or fine tune a regimen.

In some embodiments, alternative user interfaces and/or other methods of eliciting active user feedback are tested by providing different feedback methods and/or different feedback options to different users. Optionally, a particular user population is designated as a test group for a new feedback method, which optionally is iteratively updated until a stable and/or satisfactory response rate is achieved. In some embodiments, iterative A/B testing of changes to feedback methods with the whole user population or any suitable subset thereof is used until a stable and/or satisfactory response rate is achieved.

In some embodiments, determination of a satisfactory response rate is based on the relative and/or absolute rate at which an administered regimen stabilizes (or other metric of the process leading to regimen stabilization, such as regimen rate of change and/or one or more criteria for evaluation of the change) while it is undergoing active development and/or refinement; for example, as described in relation to FIGS. 1-3. For example, there are optionally a plurality of user interfaces operating to gather input data (assigned by any suitable arrangement; for example, exclusive use by particular user groups, cycled or otherwise switched for particular users, etc.). As input data is gathered, it is noted which interfaces are providing data which most affect regimen changes under some criterion or set of criteria. Optionally, the criteria include a requirement that changes made be positive (beneficial), e.g., positively associated with increasing the effectiveness of the regimen for its targeted use(s). Criteria related to rate of regimen change optionally include, for example, fastest reaching of a stable regimen (or other regimen-related target) in time, and/or smallest number of adjustment to reach a stable regimen (or other regimen-related target). In some embodiments, the regimen target is a stable regimen, after a period of initial change. In some embodiments, the regimen target is a regimen which is in some respect “preferred”; for example, elicits a higher rate of positive reports, requires a lower rate of delivery, and/or requires lower amounts of active agent(s) and/or less types of source material(s) in order to achieve a targeted effect.

Optionally, different feedback methods are provided for different user groups. For example: a feedback method relying on normal levels of fine motor control is optionally provided to one group of users. Another feedback method suited to users with a lower degree of coordination is optionally provided to users who (for example) indicate this as a preference, and/or who show signs (e.g., in collected user data) of having trouble with coordination. In some embodiments, one or more “advanced” user interfaces are made available (which may have, for example, more settings, more options for feedback, and/or more detailed regimen data). Optionally, the advanced interface is made available, for example, to users who indicate and/or demonstrate interest in taking a more active role in monitoring and/or control of their regimen.

### System Features Provided to the User

In addition to collecting data, system 400 is configured to provide information to the user in some embodiments; optionally as part of the normal operations of the inhaler 301.

### Regimens

In some embodiments, regimen options 615 are provided from server 416 to inhaler 301 as a default regimen and/or for selection. In some embodiments, regimen options 615 are developed at least in part based on analysis of inputs from a plurality of users, for example as described in relation to FIGS. 1-3.

Optionally, a default regimen option 615 is provided upon activation 602 of the inhaler 301. The default regimen option 615 may be a regimen which has been developed in response to user feedback from a plurality of users. The default regimen is optionally automatically provided as a replacement for another regimen which was previously selected, based on being a modified descendent of that regimen (the “newest version”, for example). Optionally the default regimen is a new regimen proposed by a user or a care giver. Optionally, a new default regimen replaces an older default regimen, based on a providing a better match to previously determined preferences, currently targeted effects, current patient status inputs, and/or other salient features.

In some embodiments, regimen options 615 are provided for user-guided selection during a phase of adjustment 604 prior to delivery of active agent. Selection can be by any suitable operation of a user interface 420 provided on the inhaler 301 itself, and/or using interface capabilities 412A of a dedicated and/or application-driven interface device 412. Selection is optionally limited to regimen options 615 which have been approved by a physician for a user and/or by the user in advance; and/or which are provided in order to target symptoms, side effects, and/or psychoactive effects which have been identified and/or approved for a user.

### Effect Reports and Predictions

In some embodiments, information provided to a user comprises effect reports 617 and/or effect predictions 613.

Effect reports 617, in some embodiments, comprise relatively direct responses of other users to a regimen which the current user is undergoing. These can optionally have been provided to the system 400 in the form of recorded verbal comments, written comments, and/or other indications (e.g., of effect onset, peak, and/or termination). Optionally, effect reports 617 include details of the specific regimen of a reporting user, which may, for example, be the same as that of the receiving user, or is similar thereto, or targets the same purpose.

Effect predictions 613, in some embodiments, include indications to a user of what effects from delivery of an active agent to expect, and/or when to expect them. Optionally, effect predictions are based on previous clinical and/or validation trial records. Additionally or alternatively, effect predictions are created and/or modified from baseline predictions by AI 305, based on inputs from a plurality of users. In some embodiments, the inputs comprise effect reports 617, and/or elements thereof. Accordingly, effect predictions 613, in some embodiments, comprise a form of distillation of information provided by effect reports 617. Conversely, in some embodiments, effect reports 617 from a more general user population are optionally filtered and/or modified in order to fit effect predictions 613 specific to a particular user. For example, effect reports 617 are optionally filtered so that only reports from users most similar to a current user are presented and/or reports from users having the best results (e.g. in terms of symptom alleviation, side effect intensity and/or user satisfaction) are presented. Optionally, effect reports 617 which relate to a temporal component of the experience (e.g., onset, peak and/or termination of effects due to active agent delivery) are scaled in time to match a predicted effect time course for the current user.

A potential advantage of providing effect prediction and/or report information to a user is to provide reassurance that the user's experience is substantially “normal”, and/or to help the user adjust expectations as they anticipate and/or experience effects of active agent delivery and/or to provide guidance in suggesting changes to his own regimen. Conversely, for example if there is something anomalous in a user's personal experience, the information provided can encourage a user to report the difference, allowing the user's physician and/or the AI to make adjustments to the regimen, which may apply to the particular user, and/or may become part of a regimen provided to other users.

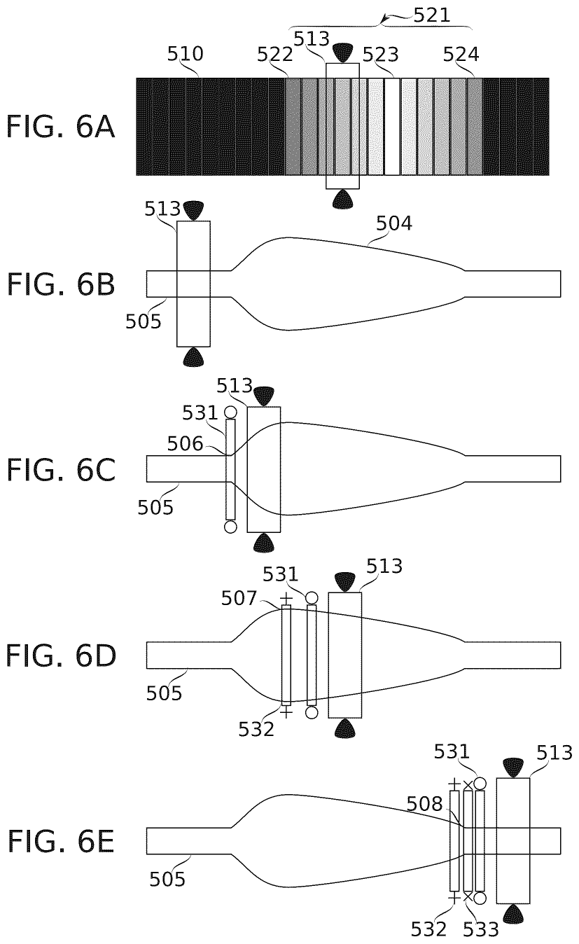

Reference is now made to FIG. 6A, which schematically represents a status bar 510 indicating a user's current (actual and/or expected) state for some effect of a delivered active agent relative to a baseline time course 521 for that effect, according to some embodiments of the present disclosure. The effect comprises, for example, pain relief, a side effect, and/or a psychoactive effect.

Time is represented in this example along with length of the bar 510. Bar 510 is optionally implemented on a general purpose pixel screen, e.g., as part of an app suitable for running on a general purpose computing device. In this example the shade (or color) of each sub-bar schematically depicts a level of a given effect. Optionally, bar 510 is implemented as a row of elements such as LEDs, addressable LCD areas, or any other suitable display device. Reticle 513 optionally represents the user's current state. Sub-bar 522 optionally represents onset of an effect, sub-bar 523 optionally represents its peak, and sub-bar 524 optionally represents the termination of the effect. Optionally, the time course 521 is moved across bar 510 as time progresses. Additionally or alternatively, reticle 513 moves as time progresses. In some embodiments other depictions may be provided including circular designs and/or text. In some embodiments a plurality of effects are presented (for example the effect on one or more symptoms and/or the degree of one or more side effects).

In some embodiments of the disclosure, information obtained from an individual current user's inputs is shown together with and/or used to modify a predicted time course.

Reference is now made to FIGS. 6B 6E, which schematically represents an effect time course graph 505, including an effect region 504, according to some embodiments of the present disclosure. In some embodiments, the width of which represents a predicted effect level which rises with increasing width (from left) reaches a peak width, and then falls again. In the example shown, reticle 513 representing the user's predicted current experience is advanced rightward along graph 515 as time 25 progresses between FIGS. 6B-6F.

At FIG. 6B, shortly after delivery of one or more active agents, there is as yet no experienced effect predicted. If all is well-calibrated, the user is able to anticipate effect onset by comparing the position of reticle 513 to the widening of the effect region 504. However, a new user and/or a regimen new to the user may not yet be calibrated. It is a potential benefit to be able to accept user input in order to introduce a user-specific calibration. Optionally, such user input (optionally received by an AI in conjunction with data describing the user; and/or data indicating other circumstances potentially having an effect on the time course) also helps an AI or other algorithm to refine the time course of effect region 504 as it is later shown to other users. Particularly for subjective measures of effect onset/maximum/termination, there may be wide differences in how users report, whether or not the actual effect time course is similar to a population-based expectation.

In FIG. 6C, reticle 513 has (in this example) passed the initial predicted rising effect phase, noted at inflection 506. A while later, the user may note (and indicate to the system) the effect beginning, and/or data from the user indicates that the effect is beginning. The system can react to this in several ways; for example, resetting the position of reticle 513 and/or rescaling bar 505. In the example shown, the system acknowledges the user input by showing a second reticle 531. As shown second reticle 531 is marked with a different symbol (a circle) as a reminder that the reticle marks the actual onset-adjusted effect prediction. Since second reticle 531 is to the left of the originally predicted experience of the user (marked by the advancing position of reticle 513), the user can see that his personal experience is developing slightly slower than the time course indicated by effect region 504 (e.g., slower than an average or otherwise defined general or “typical” user).

By the time of FIG. 6D, reticle 513 passed the predicted peak effect 507. The user continues to lag the predicted effect time course, this time by a slightly longer time than before. This shows that the rising time course of the user's experience is apparently slower than otherwise predicted, even accounting for the onset delay. Indicating this, another reticle 532 is now added, optionally marked (e.g., with a +) to remind that it indicates a maximum-effect adjusted effect prediction. In this case, the user appears to be experiencing and/or reporting the rising effect of an active agent significantly more slowly than the general population from which the time course of effect region 504 was derived. Optionally, the time of reported “peak effect” is defined as the last time at which an effect increase was reported, before any effect decrease was reported.

Finally, in FIG. 6E, inflection 508 marks the predicted termination of effect. Reticle 533, marked with an X, has been added to the figure. It appears from its position (to the right of the peak marking reticle 532) that the user's diminution time is a little faster than predicted, after accounting for the lag in reaching maximum effect. Overall, however, the time course of the user's experience is slower (since reticle 533 is still to the left of the baseline prediction represented by reticle 513).

Optionally, the next time the user initiates the same regimen, the progress of reticle 513 through the time course of graph 505 will be adjusted based on the timing of the events represented by reticles 531, 532, and 533. This can help a user set more realistic expectations about their experience. This is a potential advantage for a user seeking relief from pain and/or nausea—it can help reduce second guessing and/or anxiety as to the effect of the active agent delivery. Additionally or alternatively, if an effect really does fail to develop, for example due to a failed inhalation, a user is potentially more confident in deciding to request a second delivery.

### User-Driven Regimen Design

Reference is now made to FIG. 7, which schematically represents a method 700 for user-driven development of new regimens based on active design inputs from users, according to some embodiments of the present disclosure.

Use of machine learning (also called AI, herein) in order to develop new active agent delivery regimens and/or variations thereof is described herein, for example in relation to FIGS. 1-3. Additionally or alternatively, in some embodiments of the disclosure, active agent delivery regimens are subject to deliberate development by individual users.

In some embodiments, one or more of the categories of user-designer 721, user-tester 722, and user-standard 723 are distinguished. A user-designer 721, in some embodiments, is a user who has been granted access to the overall system 400 that allows the user at least some degree of autonomy in creating and/or suggesting particular changes to one or more regimens, and providing them to a regimen portal 730 (regimen portal 730 is optionally one of the portal services 540, described in relation to FIG. 9, herein). The user-designer 721 is optionally enabled to design and/or experiment with a regimen by using the capabilities a developer kit 711. Developer kit 711 optionally comprises a wider and/or modifiable range of source materials (e.g. botanicals), active agent types, compositions and/or concentrations, the active agents may have one or more of pharmaceutical and/or other effects (such as scent/flavoring agents and/or cognitive manipulating effects, or placebo effects, or countering effects.) which can be put together in new combinations, used to release different amounts of active agent, and or used with different timings, in order to create a new regimen—e.g., to improve on targeting of desired characteristics by an older regimen, and/or to target new regimen requirements. Optionally a user-designer 721 is provided with a developer kit 711 that may be controlled manually (at least partially).

In some embodiments, materials provided with a developer kit 711 as a basis for development of regimen variations include varieties of one or more of the following botanical and/or botanically derived materials, optionally as raw botanical material: Cannabis strains, terpenes, cannabinoids, monoamine oxidase inhibitors (MAOI), medicinal botanicals (optionally combined and/or combinable with any suitable selection from the cannabis strains, terpenes and/or cannabinoids), other medicinal botanicals (optionally combined and/or combinable with any suitable selection from the cannabis strains, terpenes and/or cannabinoids), alkaloids, nicotine and tobacco.

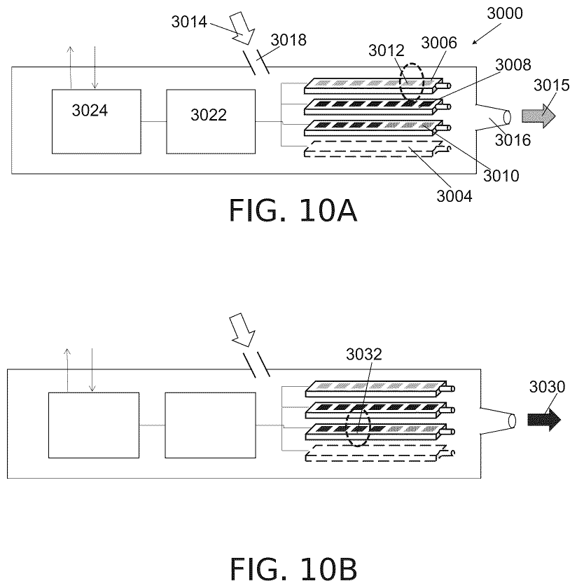

In some embodiments, providing of a wider range of materials to a developer, tester, or standard end-user is enabled by the use of cartridges comprising a plurality of individually addressable dose sections. Examples of such a cartridge are described in relation to FIGS. 10A-14B herein. In some embodiments, a magazine may contain a plurality of multi-section cartridges. Optionally, multi-section cartridges are provided separately from magazines. Optionally, multi-section cartridges are used with delivery devices configurable to hold and activate sections of a plurality of different cartridges as part of a single dose delivery.

Optionally, developer kit 711 includes support for making use of materials not yet a part of the standard supplies available from manufacturer or manufacturers 701; for example, equipment allowing cartridge loading. As manufacturing under non-laboratory conditions even for development purposes, is potentially difficult to control and transfer to a manufacturable metered-dose product, it is anticipated that many important self-manufacturing uses of a developer kit will not be by pure “user-developers”, but rather developers who work for actual and/or potential suppliers (for example third party suppliers, which are discussed and defined in relation to FIG. 9, herein). However, for some types of source materials, well-controlled load-out may not be crucial; for example, where botanical effects are relatively safe, e.g., sensory effects (smells, tastes). Optionally, there is a distinction made between a full developer kit 711 that allows cartridge manufacture (custom loading), and limited developer kits 711 that simply have a wider and/or more flexible than normal range of actual and/or optionally supplied botanical load-outs.

Optionally, developer kit 711 includes cartridges designed to allow cleaning and reuse by a qualified user-designer 721. Optionally, self-identification mechanisms for developer kit 711 supplies are designed to be at least partially modified by a user-designer, in order to allow freer experimentation. In some embodiments, user of developer kit 711 components is monitored by regimen portal 730 in order to identify and/or restrict potential abuse (such as unauthorized load-outs of active agent). Optionally user-designer 721 may provide a new regimen without experimentation (for example, based on scientific knowledge and/or prior experiences and/or when dealing with recreational use and/or when no significant side effects are expected. Such experimentation free design may be subject to strict limitations (e.g. dose per inhalation or per period of time for a given active agents, and/or approval by a qualified physician).

Once a user-designer 721 has a new regimen ready for testing, in some embodiments, details of the new regimen are uploaded to regimen portal 730. For example, the new regimen optionally is new in at least one of: its combination of source materials, use of a particular model of active agent delivery device, use of scent and/or flavor agents, new combinations and/or ratios of active agents, inter-dose timings, dose amounts, and/or monitoring via the delivery device selected.

Optionally, one or more human administrators (administration 740) reviews new regimens for appropriateness and/or potential value, before the new regimen is made available for any wider group of users.

In some cases, a new regimen requires no new consumables and/or arrangements thereof; and can, accordingly, be directly provided to a group of one or more user-testers 722 for validation, used with an existing standard SKU (stock keeping unit) product 713. User-testers 722 are optionally selected by general active recruitment and/or spontaneous voluntary sign up (e.g., via regimen portal 730), and/or active recruitment for users meeting specified criteria (e.g. a shared symptom or condition and a shared purpose).

Optionally, a tester kit 712 is produced, or already exists, which is particularly manufactured for purposes of testing new regimens. Optionally, a tester kit “expansion pack” is specially manufactured for the requirements of a new regimen; for example, if the new regimen relies on cartridge capabilities which are not already available. Optionally, use of a tester kit comprises loading an inhaler device with a combination of multi-section cartridges selected from a variety of previously available multi-section cartridges, each variety with its own source material composition. Sections of a cartridge optionally comprises the same or different source materials, amounts of source materials, and/or combinations of source materials and/or active agents. A tester kit 712 will typically not provide any capability for loading or re-loading cartridges with material. A tester kit 712 may also be more restricted in the range and/or flexibility of materials available than a full developer kit 711. This is practical, since a baseline regimen was already defined.

Compared to a standard stock keeping unit (“SKU”, e.g., a standard magazine offered to sale for users), a tester kit 712 optionally provides more flexibility for magazine loading; e.g., user-testers may be enabled to load out their own magazines according to regimen protocol requirements. Optionally, user-testers 722 are provided partial regimen parameter control that standard users lack, for example, allowing them to suggest and try out minor regimen modifications. Optionally, new regimens provided to user-testers 722 are deliberately spread out over a larger range of related regimens than would be usually provided for use with a standard SKU 713; for example to speed up AI-based learning of optimal regimen parameters (which, optionally, also takes place during this phase of regimen deployment). Optionally, at least some user-testers 722 are provided with full or restricted-type developer kits 711 (for example, if no adequate tester kit 712 is presently available). Such user-testers are optionally instructed, at least for testing purposes, to restrict their use of the greater flexibility of such a developer kit 711 to testing, not development. Optionally, user-testers 722 are provided with tester kit 712 having standard capabilities, and tested by actual use of the regimen.

Regimen test results are optionally monitored through regimen portal 730. At some point, e.g., upon a decision by human administration 740, a standard SKU 713 is optionally released from manufacturing 701. The decision to do so is optionally based on considerations of safety, popularity, user satisfaction, business considerations, and the like. The standard SKU 713, in some embodiments, encapsulates in a single sealed magazine lessons learned during the design and testing phases, so that standard users 723 are able to use the standard SKU 713 directly throughout the predicted range of usage parameters, while minimizing and/or removing complications such as cartridge load-exchanging. Optionally the standard SKU 713 comprises instead of a sealed magazine, a kit of multi section cartridges sold in unison (sealed together and/or separately). In some embodiments, AI-based regimen modification continues even beyond full release of the regimen for use by the general user base. This potentially helps, for example, with fine tuning for user types not well represented in the testing population, detecting and/or reacting to supply chain variability, integrating minor changes affecting the general population of users, etc.

Similar to the situation with developer kits 711, standard users 723 are optionally allowed access to at least certain purchasable tester kits 712. Optionally, tester kits 712 can help a new user decide what standard SKU 713 they should switch to. Tester kits 712 can also be useful for standard users who believe that they have needs not yet met by standard SKUs; granting a degree of experimental freedom, while still remaining within relatively safe bounds. For example, in a generally purchasable tester kit 712, botanicals are still manufactured under controlled circumstances, cartridges well-identified, and safety checks meant to detect improper use optionally remain intact.

### Uses of Machine Learning Results

Reference is now made to FIG. 8, which schematically represents relationships of machine learning to other aspects of a system 400 for adaptive operation of an inhaler 301 to some of the inputs to and results of machine learning, according to some embodiments of the present disclosure.

Original input data sources in the system 400 are represented in FIG. 8 as comprising users 802, and devices 804 (that is active agent delivery devices such as inhaler 301, and/or auxiliary hardware such as interface device 412).

At the next level of FIG. 8, inputs are shown divided into four categories: command 812, reports 814, regimen info 818, and all other input 816. Regimen info 818 may include data from sensing devices 421, which accumulate and report actual sequences, timings, and/or other settings used with an inhaler 301 during operation of the device 804 to delivery active agent. Other input 816 may include data from any other sensors, and also non-verbal inputs from user 802 (optionally excluding communicative symbolic—but not necessarily “verbal”—inputs such as typed words, emoji, emoticons, etc.). Regimen info 818 and other input 816 are shown as being processed by general input processing 824, which can include any form of preprocessing applied (e.g., to extract salient features of the input) before being supplied to machine learning at block 826.

### Natural Language User Interfacing

In some embodiments of the disclosure, users are able to provide written and/or spoken input to system 400, for example via a microphone 423. Optionally, input is given as typed words or other symbolic information, for example via a touchscreen and/or keyboard. For purposes of discussion, such inputs are divided into commands 812 and reports 814.

Commands 812, in some embodiments, are optionally provided in the form of natural language statements, which are not necessarily commands in the sense of explicit instructions. Nevertheless, the statements are optionally interpreted as conveying restrictions and/or conditions which the system should apply to the selection of a regimen that sets the delivery of active agent to the user. Examples include:
“I'm having nausea.” (optionally understood as describing a symptom to be treated).
“I need to exercise in an hour.” (optionally understood to set an activity context; regimen is to be adjusted so that the user will be free of effects in an hour)
“But I keep getting headaches.” (optionally understood as a side-effect adjustment request; optionally, this is additionally treated as a report 814, as later described).
“Just something for the nausea.” (optionally understood as a request to limit to a treatment effect, avoiding recreational, e.g. psychoactive, effects)
“The one that tastes minty” (optionally understood as a request to include a flavoring agent in the regimen)

Optionally, as each command 812 is received, the system converts the statement into a selection criterion, based on available natural language processing tools 822, optionally augmented by machine learning 826. In some embodiments, the user receives in return an indication of what the system understands the user to have requested, optionally along with indications of changes to the current regimen selection (and/or available regimen options). These indications are optionally spoken aloud by a machine text-to-speech facility, shown on a screen as text, and/or expressed as changes to available options available from on-screen lists, buttons and/or other graphical user interface elements. If there is no way to respond to the request, the system optionally indicates that. Optionally, the system indicates what it understands to be the “best it can do”.

As for the implementation of this, the field of natural language user interfaces has achieved several widely-deployed implementations capable of speech to text conversion, together with assignment of sufficient meaning to the extracted text to allow machine-supplied responses. Simplifying greatly, there are optionally two main levels to implement: conversion of speech to recognized words, and extraction of meaning from the words. These two levels may be interlinked, since recognition can be informed by likely meanings. Speech to text implementations are widely available, with accuracy scores likely to increase for uses where use of a limited vocabulary of key terms can be assumed.

Regarding meaning, in the case of active agent delivery devices, a user will potentially relate actions to him/herself and his/her symptoms, while what the system is actually controlling is selection and administration of active agent, filtered through a selection of regimen options.

In some embodiments, this circumstance is overcome at least in part by instructing users to use some form of controlled natural language, and making a mapping that converts user-centered commands to device-centered results. For example, the meaning behind a potentially ambiguous command 812 such as “just something for the nausea” would instead be expressed more formally, e.g., in a verb-noun-qualifier format such as “treat nausea only.” Since the only thing the device can do to “treat” is administer active agents, the general action required is potentially clear. The object “nausea” optionally then can be treated as a search request for a regimen that has been marked for “treats nausea”, with the “only” understood as a limiter to avoid regimens marked as having other effects.

Using machine learning techniques, such analysis need not be carried out in detail for every possible circumstance. Optionally, even specification of a controlled natural language is dispensed with, since the domain terminology (operation of a particular device for a few particular purposes) may be quite restricted compared to open-domain natural language usage. In some embodiments, a large number (e.g., 100, 1000, 5000, or another larger, smaller or intermediate number) of sample commands 812 (from actual and/or simulated device use) are annotated by human scorers, and used as a training set for one or more machine learning algorithms. As failed responses to new inputs are noted by human review, these inputs can also be annotated and fed back into learning set, so that accuracy potentially increases over time and with increased use. Optionally, machine learning takes into account not only commands and actions, but also additional available inputs, which potentially affect context.

In contrast to commands 812, reports 814 are more likely to include a wider variety of statements and/or expressions, for example as users creatively translate subjective experiences into words. For purposes of this description, “reports” are considered to comprise of any text (words or otherwise) and/or recorded speech which can be converted to text (e.g., by NLP input processing 822), but which cannot be understood as a command 812. Even among commands 812, some examples may also sever as reports; for example if a user requests “more” or “less” of some effect, there is some implication that a previous experience had too little or too much of that effect. Optionally, moreover, there is a facility provided (e.g., a mode selector) for explicitly indicating which expressions are commands and which should be taken as reports. Optionally, reports are associated with time-stamps, which are optionally indexed relative to times of device usage.

The more open content of reports potentially interferes with full machine-implemented interpretation of their meaning (although speech-to-text may still be implemented, in some embodiments, with a potentially high degree of accuracy using commonly available tools). In some embodiments, report contents are analyzed statistically, e.g., for the occurrence of certain words and phrases. Words and phrases that occur often—particularly those which have meaning already known to be relevant to active agent delivery and/or the effects thereof—are treated as representing a “trend”. Optionally, trends need not be expressly linguistic; for example, text reports comprising emojis of faces with different expressions are optionally understood to convey positive or negative results according to the expression.

### Machine Learning

At block 826, in some embodiments, machine learning algorithms (also referred to herein as AI) operate on provided input. Several types and purposes for machine learning are described herein, including regimen adjustment 827, information presented back to users 825, alerts 828 to administration personnel 832, and/or reports 830 provided to administration personnel 832 and/or to users in response to queries. In some embodiments, administration personnel 832 use outputs received as a basis and/or trigger for further activities: for example business activities such as research and development, manufacturing, marketing, and/or quality assurance; and/or clinical activities additional and/or alternative to regimen adjustment 827, such as user counseling.

A non-limiting list summarizing some more specific examples is now provided, including:
Using regimen info 818, other input 816, and/or reports 814 from a plurality of users in order to evaluate a regimen for its effects.
More particularly, detecting trends (indicated by the same inputs as just listed) among a plurality of regimens in order to suggest a new regimen which appears likely to achieve a targeted range and/or balance of effects.
Suggesting for a particular user an optimal regimen, based on inputs from the user, and based also on regimen results known from a plurality of other users to whose inputs the current user's own inputs can be related.
For a supposedly fixed regimen used repeatedly over time by a plurality of users, detecting trends (particularly but not exclusively based on changes in word and/or phrase contents of user reports 814) that potentially indicate a change in effects—for example due to an issue with growing, manufacturing, storage, and/or shipping.
Using trends in reports 814 to identify unexpected issues with a regimen as such (especially near the beginning of its distribution), e.g., reports mentioning nausea for a regimen that was not expected to induce such a side effect.
System-initiated research and/or case studies regimens to validate specific patient feedback on a plurality of patients. For example, AI detects a potential trend, but the data is insufficient for confirmation thereof; and/or a new source material, a new active compound and/or a new purpose of use is introduced. The system may initiate a study to obtain the missing information. For example, a plurality of patients having similar personal properties and/or diagnoses are selected and provided with one or more regimens to test the suspected trend. Optionally in the framework of such a study, some of the users are provided with placebo instead or in addition to active agent(s). When a placebo is used, the information may be restricted in a blinded or double blinded manner so as not to affect the results.
Converting other input 816 and/or reports 814 from a plurality of users into population summaries which can be provided back to individual users, e.g., to help individual users relate their own experiences to the experiences of the user base as a whole or in any relevant part.
Alerting an administrator to an unexpected change in input data gathered from one or more ongoing regimens.
Alerting one or more administrators (optionally including a physician involved in treatment of a user) of a potential for a user being involved with substance abuse using the monitored active agent delivery device. In some embodiments, the pattern of substance abuse is indicated, for example, by continuing usage of “emergency” dosing requests, even after one or more initial adjustments of the baseline regimen have been made.
Alerting one or more administrators (optionally including a physician involved in treatment of a user) of a potential for a user to develop a pattern of substance abuse or an addiction. In some embodiments, machine learning examines, for a plurality of users, trends in and/or features of otherwise legitimate patterns and/or medical background related information of inputs leading up to a determination that those users later converted to abusers of the substance. Optionally, other users who reproduce those trends are identified before evidence of actual abuse occurs, potentially allowing a pattern of abuse to be averted.
For an identified actual or potential abuser, in some embodiments, switching the user's regimen over to a new regimen targeted at reducing or preventing addiction, and/or easing withdrawal. For example the new regimen comprises immediate and/or tapering reduction of active agent released in regimen doses, and/or substitution to a less harmful or less addictive substance in some or all regimen doses and/or administration of anti-addictive active agents (e.g. agents that are known to assist in the withdrawal process and/or prevent addition) and/or modifying a sequence and/or ratio between a potentially addictive agent and an anti-addictive active agent. In some embodiments, a regimen targeted at reducing addiction and/or easing withdrawal is developed based on input data sourced from a plurality of users, for example using methods of and/or systems for input collection and/or input processing described herein.

### Platform Customization Capabilities

Reference is now made to FIG. 9, which schematically represents aspects of system 400 of FIG. 4 which are optionally available for customization by users and/or suppliers, for example platform licensees, according to some embodiments of the present disclosure. Although users may also be considered third parties with respect to each other, herein the term “third party supplier” is used to indicate suppliers operating within the framework of system 400 at a commercial scale (whether or not for profit) other than an original and/or primary supplier of supplies and components of system 400, and/or other than a holder of controlling rights with respect to such activities; either defined according to commercial conditions and/or agreements that may become established.

### Customization of Delivery Materials and Regimens

Block 902 represents a user's interface to the rest of the system 400, including an active agent delivery device (optionally inhaler 301), and optionally including a network-connected interface device 412. It should be understood that there are optionally a plurality of designs of active agent deliver devices available for use in the system 400, each comprising its own particular features, while still enabled to interface to relevant elements of the system 400, substantially as described for inhaler 301.

In some embodiments of the disclosure, materials and supplies for delivery of active agent can be customized by participants in the “ecosystem” of system 400. For example, cartridges 403 and/or magazines 402 are optionally available in variations from original-manufacturer designed configurations.

In some embodiments, selected suppliers are optionally provided with rights and identifiers within system 400 that allow them to prepare active agent delivery materials such as cartridges 903 and/or magazines 909, and to mark these manufactures in a way (e.g., provision with machine-readable IDs and/or self-descriptive data) which can in turn be recognized by devices of block 902. The suppliers optionally provide regimens 931; these may be accessed via information carried by cartridges/magazines themselves, and/or via portal services 940. In some embodiments, development by suppliers (e.g. third parties) is supported within the system by developer kits 711, for example as described in relation to FIG. 7. Optionally, suppliers are provided access to analytics feedback regarding use of their supplies (e.g. from the portal services 940), derived from any suitable information collected by system 400. Suppliers may use the information, for example, to optimize production, guide research and development, target marketing, etc. Optionally, suppliers are provided access to user feedback-driven regimen refinement and/or development capabilities of system 400, for example as described in relation to FIGS. 1-3.

In some embodiments, certain users (for example, user-designers 721 of FIG. 7) are enabled (e.g., given appropriate access rights to tools implemented by portal services 940) to design user-developed regimens 932 based on existing cartridges/magazines; and/or to design and/or suggest user-customized cartridges 905 and/or user-customized magazines 911. Access to user-designed regimens and/or ordering of user-customized cartridges/magazines is optionally opened to a user base in stages, for example as described in relation to FIG. 7.

### Customization of Software (Plugins)

In some embodiments of the disclosure there is defined one or more software plugin interfaces allowing expansion of the capabilities of framework software of system 400 by users and/or suppliers. Plugins 920 optionally comprise software modules which can be run on any appropriate computing device which is a part of system 400. It is anticipated that plugins 920 will ordinarily run on client-side devices such as interface device 412, but in some cases it may be appropriate to provide a server-side plugin implemented by computers comprising portal services 940.

Examples of plugins include, for example, plugins 921 which can be used for assessment of user status, and/or active agent effects on a user. Such plugins could be provided, for example, in order to allow patients with disabilities to provide input to system 400 in ways other than those enabled by default system interfaces. Suppliers of regimens 931, cartridges 903 and/or magazines 909 optionally may provide plugins supporting input requirements other than those supported by the default system. Users themselves (e.g., user-developers in the community of users; optionally including individual developers who may not be themselves users, but have motivation to help users) are optionally enabled to design plugins which provide ways to assess aspects of experiences (state and/or active agent effects) in ways different from assessments supported by default interfaces.

In some embodiments, a plugin interface for experience app plugins 922 is optionally provided. In some embodiments, experience app plugins 922 have as a main function some form of engaging the user during waiting and/or altered experience periods associated with use of the inhaler (e.g., while waiting for effects to begin, peak, or terminate, and/or while experiencing those effects). An experience app 922 may, for example, combine features of the effect reporting/predicting described with reference to FIGS. 6A-6E with different graphics or sounds. Additionally or alternatively, the experience app 922 takes the form of a task (optionally framed as a game). In some embodiments, the plugin interface allows data indicating user interactions with the task/game to optionally be provided as input provided to portal services 940. Such input is optionally used in the modification of regimens, for example by an AI algorithm as described in relation to FIGS. 1-3. Optionally, an experience app plugin 922 is provided at least in part to serve as a distraction, with or without associated reporting; e.g., to take a user's mind off of pain and/or nausea while waiting for pharmaceutically active agent effects to take hold. In some embodiments, the experience app plugin 922 provides sensory enhancement to active agent effects, for example, by offering visual and/or auditory stimuli to accompany psychoactive effects. Optionally, an experience app plugin offers reassurance or assistance to a user; for example, by making it easier for a user to connect with (e.g., via video, telephone and/or a social network) a friend, relative, and/or care provider during use of inhaler 301.

In some embodiments, a plugin interface is provided to allow customization of the process of regimen selection (regimen selection UI 923). Such plugins optionally change the regimen selection process to potentially better suit the preferences of an individual user. For example, a plugin may be relatively experimental and/or informative in approach (many options and/or more detailed display of available data from other users). Alternatively, a plugin is optionally streamlined to provide greater simplicity, and/or specialized to be navigated by users with particular impairments. Optionally, regimen selection plugins help streamline access to information specific to a particular community of users; for example community postings regarding questions and answers, guides, new products, availability updates, and the like.

While the above types of plugins have been described for operation in conjunction with particular phases of the use of an inhaler 301 (or other active agent delivery device), it should be understood that such phase-specific descriptions are exemplary only, and should not be considered as limited to those phases only.

### Input-Driven Customization

In some embodiments of the disclosure, a portal service 940 of a system 400 is configured to accept one or more types of “arbitrary content” user input (user free-form expression 933); comprising, for example, words, sounds, character combinations, icons, and/or images. Examples of such inputs from the realm of social media include hashtags, emoticons, emoji, “meme” images, musical and/or sound samples, and the like.

In some embodiments, such inputs optionally comprise self-photographs, wherein content is conveyed by, for example, a user's facial expression, skin coloration (or change therein, e.g., blushing), and/or state of pupil dilation. Optionally, facial expressions are natural. Optionally, facial expressions are exaggerated by a user for expressive effect, for example, tongue out to express nausea, a grimace to express pain, a large smile to express contentment, etc. Optionally, photographs are converted based on facial expression-recognition algorithms (optionally run on user-local computational hardware) to non-identifying icons (e.g., corresponding emoji expressions) before being shared, which has the potential advantage of preserving privacy.

In some embodiments, image data of the user (and particularly, in some embodiments, facial expressions of the user) are obtained autonomously during use of an application for interacting with system 400, or semi-autonomously (e.g., upon specific user activation). Optionally, baseline images of the user are obtained and stored before treatment and/or periodically and/or before delivery of an active agent thereby to define a baseline for analysis. In some embodiments, image data are used to detect the user's status (e.g., affective state; particularly, in some embodiments, with respect to pain or nausea apparent in a user's facial expression) before and/or after delivery of a substance. Optionally, a user status change indicates effects of the substance. Optionally, functionality is embedded in an application that initiates video recording on a smartphone while a user is submitting dose feedback; alternatively an array of static photos taken from the smartphone front camera is acquired. Images are optionally pre-processed locally (e.g., to extract key frames and/or features, and/or reduce image size) before images and/or data extracted therefrom (for example, reduced to representations of facial feature position metrics, and/or converted to a wireframe representation) are uploaded to a remote server or server array (optionally centralized or distributed). Conversion of images to reduced form (wireframe and/or facial feature position metrics, for example) has the potential advantage of reducing a risk of anonymity loss for a user.

In some embodiments, the video or image array uploaded is compared with a large database constructed, e.g., by a status/emotion recognition service provider. The database is optionally constructed, for example, using neural signal processing, computer vision and/or deep learning on images of live subjects, optionally by association of a training set of live subject images to emotions indicated, or by another machine learning training technique known in the art. User's face expressions are scored according to comparison results, and a value is returned as input, representing a position on an emotional scale.

Additionally or alternatively, in some embodiments, a similar chain of processing is performed for voice recordings (e.g., recording, optional pre-processing, comparison with a database of scored reference recordings, and reporting of a scale value). The scale values are optionally with respect to, for example, emotions detectable in the voice data, and/or to vocal evidence of impairment (slurring, shaking, and/or slowness, for example).

In some embodiments, a service directed to detection of a particular disease from image and/or voice data (e.g., early detection of parkinsonism via tremor detection) is used. Optionally, any other available data is used in such remote diagnostic and/or mental state assessment processing, for example, pressure sensors and/or motion sensors.

In some embodiments, users optionally use these inputs to indicate meanings related to use of an inhaler 301 (or other active agent delivery device). The meanings are optionally relevant to functional aspects of use; though they are possibly colloquial, oblique, and/or humorous in expression. Such inputs may related, for example, to indicate problems with a regimen such as side-effects (e.g., #headache, #tootired, #HUNGRY, etc.), notable positive features of the experience (e.g., #clearhead, #lowanxiety, etc.), and/or apparent problems or impatience in achieving desired effects (e.g., #stillhurts, #wheresmybuzz, #gr8nowimsickANDhigh, etc.). In some embodiments, the input itself may be at least superficially insignificant and/or unintelligible in the context of regimen effects, but nevertheless be indicative of a trend relevant to device use. For example, users of a specific regimen might all begin sharing the same image, while others do not; this might be indicative (for example) of a particularly well-interconnected, upset, or activist community, depending on content and context.

In some embodiments of the disclosure, one or more techniques of AI (machine learning) are applied to detect trending expressions (in the general sense of words, symbols, images, sounds, etc.). Optionally, the AI applies dictionary matching and/or technique, such as a NLP technique (e.g., as described in relation to FIG. 8), to try to determine the valence (positive or negative) and/or content (e.g., related to nausea, pain, psychoactivity, etc.) of the input. Optionally, a human administrator reviews candidate trends in order to help assess the trend's meaning, and/or whether the trend rises to the level of an action item. For example, expressions of nausea which become increasingly associated with a regimen after a recent modification potentially indicate that the change caused (or perhaps decreased, depending on valence) a nausea side-effect. This may indicate that user warnings should be changed, that the regimen change itself should be reverted, that a limit to further changes to the regimen should be imposed (e.g., no more of a suspected side-effect inducing component should be added to the regimen), that adding an antagonist to the side-effect should be prioritized, and/or another action item may be generated. Expression changes associated with a particular production lot potentially point to a manufacturing, shipping, and/or storage irregularity. Optionally, human administrator clarifications and/or corrections are fed back into the AI's learning database, potentially helping to improve the identification and/or characterization of subsequent expression trends and/or risk factors.

False positives: It should also be understood that some expression trends are potentially unconnected to device use, e.g., arbitrary fads and/or memes among the user base, and/or expression trends reflecting something in the wider culture unrelated to device use. In some embodiments, an AI (machine learning) algorithm is trained to recognize and optionally discount and/or flag trends which appear to be simply mirroring trends which it also detects (and/or it has been otherwise informed are existing) in the wider culture. Wider culture input is optionally based, for example, on the collection and/or analysis of general social media content made available, for example, by a third party. Arbitrary fads and/or memes may potentially be identified by a lack of correlation with particular regimens and/or changes thereto; or simply because they are known to be irrelevant by a human administrator who flags the arbitrary trend accordingly.

Cartridges with a Plurality of Source Material Sections
Inhaler and Cartridges with a Plurality of Source Material Sections

Reference is now made to FIGS. 10A-10B, which schematically illustrate an inhaler device configured to receive a plurality of source material cartridges, according to some embodiments of the present disclosure.

In some embodiments, inhaler device 3000 comprises a plurality of recesses, slots, receptacles, connectors and/or other structures each configured for receiving one or more source material cartridges. In the example shown, device 3000 comprises an empty receptacle 3004 and three source material cartridges 3006, 3008, 3010 received within three respective receptacles.

In some embodiments, each cartridge comprises a plurality of source material sections 3012. In some embodiments, for example as shown in cartridge 3006, the plurality of source material sections include identical content, for example including the same plant or plant compositions and/or the same active substance or compositions of active substances, Alternatively, for example as shown in cartridge 3010, different source material sections comprise different content, for example different plants or plant compositions and/or different active substance or compositions of active substances.

In some embodiments, a specific cartridge and/or a specific source material section within a cartridge is addressed according to a predefined regimen. Additionally or alternatively, a specific cartridge and/or a specific source material section within a cartridge is addressed upon demand, for example selected by a user according to their needs and/or desires. In this example, FIG. 10A illustrates selecting of a source material section from cartridge 3006; FIG. 10B illustrates selecting of a source material section from cartridge 3010.

In some embodiments, during use, source material of one or more selected sections is heated (for example using a heating element such as described hereinabove), and air 3014 that was drawn into the device and/or otherwise entering the device (for example via tract 3018) is allowed and/or directed to flow through the material of the selected section. Air 3015 imbued with the released active substance(s) is then delivered to a user via an output 3016 of the device (for example comprising a mouthpiece). In some embodiments, air imbued with the active substance exits the inhaler in response to inhalation of the user. Additionally or alternatively, the device includes a fan or source of pressurized air (not shown) that can augment and/or replace the force of inhalation of a user.

In some embodiments, the selected sections differ in content, so that different active substances or compositions thereof are released from each of the sections to be delivered to the user. For example, in FIG. 10A, air 3015 imbued with the active substance(s) released from source material section 3012 exits the inhaler device to be delivered to the user; in FIG. 10B, air 3030 imbued with a different active substance and/or different composition of active substances (as compared to the active substance or composition thereof released from section 3012 in FIG. 10A) released from source material section 3032.

In some embodiments, device 3000 comprises a controller 3022 configured for controlling selection and/or access to one or more specific source material sections and/or access to a selected cartridge. Optionally, two or more sections are accessed simultaneously to obtain a selected amount of active substance, a selected composition of active substances, and/or a selected effect on the patient. Optionally, different airflow and/or heating regimes are used for each of a plurality of sections accessed simultaneously, thereby affecting the composition or proportion of active substances in the airflow towards the user.

Optionally, the controller selects sections that are identical in content. Alternatively, the controller selects sections that differ in content. Optionally, the controller selects sections from the same cartridge. Alternatively, the controller selects sections from different cartridges.

In some embodiments, controller 3022 is configured for selecting source material sections for use according to a predetermined order, for example according to a regimen.

Controller 3022 is configured for receiving data from one or more sensors indicative of one or more of airflow within a section, temperature at a section and humidity are used as feedback to control the extraction and determine the amount of active agent(s) extracted from the section.

Additionally or alternatively to a controller, device 3000 comprises manual control (for example an actuator such as a slider) configured for selecting and/or enabling heating and/or airflow access to a selected section.

In some embodiments, the controller and/or manual control are configured to allow “mix and match” of different source material sections according to their content. Optionally, when one cartridge is depleted or a change or addition of a source material is included in an adjusted regimen, the user is instructed to replace and/or insert a defined cartridge to the device.

In some embodiments, multiple source material sections having contents that differ from each other are selected in order to treat a certain medical condition. Examples of active substance compositions and the medical condition that can potentially be treated using said active substances may include: active agents that provide a synergistic effect, active agents that provide the same effect but each with different advantages or disadvantages, active agents that potentiate or attenuate other one another or other active agents (e.g., alter the effective therapeutic window or therapeutic index of one-another), active agents that provide contradictory effect, such as, for example, THC is counteracted by CBD, and active agents that have counteractive but desired effects and need to be spaced apart.

Some active agents' combinations, which can be effectively delivered using the devices provided herein, according to embodiments of the present disclosure, include, without limitation, nicotine and THC, caffeine and THC and CBD and THC. Some combinations are intended for recreational use, and may include combining or changing between different tobacco blends, tobacco having different added active substances, different cannabis strains or blends, different plant material for other plants and any combination thereof.

In some embodiments, selection of multiple source material sections is performed to accurately control the amounts of active substance(s) provided, for example by using different source material sections that include different amounts of active substance. For example, a first source material section including 5 mg of an active substance can be delivered along with a second source material section including only 1 mg of an active substance, to reach a precise total amount of 6 mg active substance.

In some embodiments, device 3000 comprises a communication module 3024. Optionally, communication module is configured for sending and/or receiving data from one or more of a user input device; a database; a memory; an online data source; a physician; and/or others. Optionally, selection of source material section(s) for use is performed according to instructions received via the communication module. In some embodiments, data received from user input device comprises feedback regarding the effect of treatment. Optionally, the feedback is collected by one or more sensors of the user input device. In some embodiments, treatment or use for any other purpose is controlled (for example adjusted from a predefined regimen) according to the received feedback data.

Example of Cartridge with a Plurality of Source Material Sections

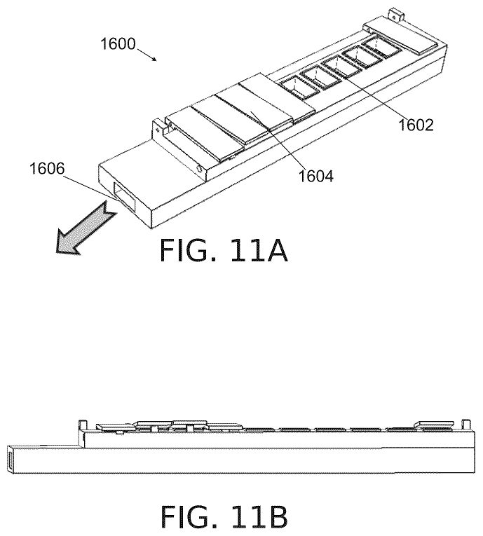

Reference is now made to FIGS. 11A-11B, which respectively show an isometric view (FIG. 11A) and a side view (FIG. 11B) of an arrangement of source material sections structured to provide for separately accessing each of the plurality of source material sections, according to some embodiments of the present disclosure.

In some embodiments, arrangement 1600 comprises an array of source material sections 1602. Optionally, the sections are linearly aligned with respect to each other, for example as shown herein. It is noted that other arrangements and/or spatial distributions of the plurality of sections are also contemplated. In some embodiments, each section 1602 can be accessed by lifting, shifting and/or otherwise moving a cover 1604 that blocks passage of air to and through and source material of the section. In some embodiments, movement of cover 1604 is actuated magnetically, manually, and/or electrically. In some embodiments, cover 1604 is pivotably coupled to a hinge.

In some embodiments, air that flows into an opened source material section passes through the source material and into a conduit (an opening of which is shown at 1606) to be delivered to a user. Optionally, the conduit is a shared conduit for the plurality of source material sections.

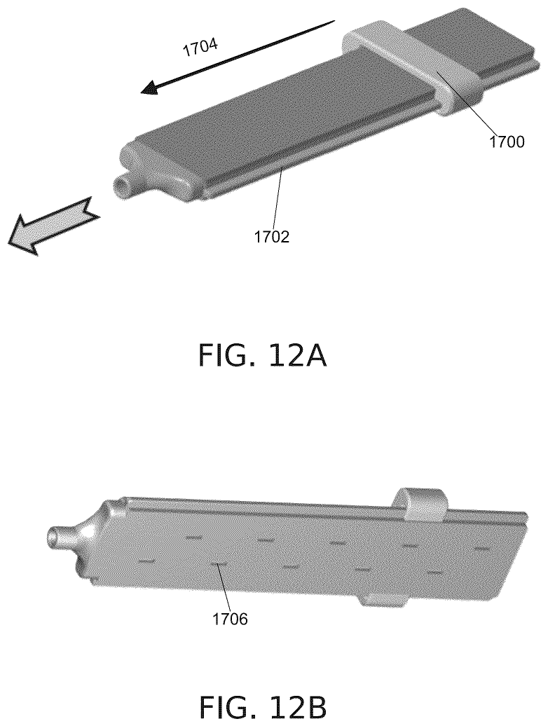

Reference is now made to FIGS. 12A-12B, which illustrate a slidable actuator 1700 configured for unblocking at least one airflow path associated with at least one source material section and/or for activating a heating element associated with the at least one source material section, according to some embodiments of the present disclosure.

In some embodiments, actuator 1700 is moved along housing 1702, for example slid along a long axis of the housing such as in the direction illustrated by arrow 1704, to provide for accessing one or more selected source material sections underlying housing 1702. In some embodiments, actuator 1700 is configured to shift a cover (such as cover 1604 as described hereinabove) of an underlying source material section.

In some embodiments, actuator 1700 is configured to activate a heating element associated with a source material section, for example by electrically coupling the heating element (such as an electrically conductive mesh) to a power source, e.g. a battery.

Additionally or alternatively, in some embodiments, electrical coupling is actuated in response to inhalation. Optionally, an electrical circuit is closed in response to sensing of a flow related parameter (e.g. pressure or a change in pressure). Additionally or alternatively, an electrical circuit is closed by movement of a flap, valve and/or other mechanical element that shifts in response to airflow.

In some embodiments inhalation acts as a trigger for a physical or electronic sensor such as but not limited to conductivity of the lips, temperature of the lips, change in pressure due to inhalation, characteristic accelerations caused by motion with reference to the earth's gravity, proximity sensors and/or light sensors.

In some embodiments the actuator is used to connect electrical circuitry to perform aforementioned coupling such as via pogo-pins, leaflet connections, direct galvanic connection and/or others.

In some embodiments the electrical heater is actuated based on determining a change in airflow for example as described in International Patent Publication No. WO2013060784, which is incorporated herein by reference, are also contemplated by this application. For example, any sensor which can detect airflow may be used. The sensor may be an electro-mechanical device. Alternatively, the sensor may be any of: a mechanical device, an optical device, an opto-mechanical device, a micro electro mechanical systems (MEMS) based sensor and an acoustic sensor. The sensor can be a thermal conductive flow sensor, a pressure sensor, an anemometer. Optionally, the sensor may be able to not only detect airflow but also be able to measure it. The sensor may be configured to deliver an analogue electrical signal or digital information that is representative of an amplitude of the airflow.

In some embodiments, movement of actuator 1700 is performed manually (e.g. by a user). In some embodiments, a user advances actuator 1700 prior to and/or during usage of the device. A potential advantage of a user controlled actuator may include allowing the user to control the rate and/or duration and/or amount or active substance delivered.

Alternatively, movement of actuator 1700 is performed automatically, for example according to a predefined protocol.

FIG. 12A illustrates a first side of the device across which actuator 1700 is advanced; FIG. 12B illustrates a second, opposite side of the device, comprising a plurality of slots 1706 through which air enters the device. Air entering the device may enter source material sections upon unblocking of associated airflow paths (not shown in this example).

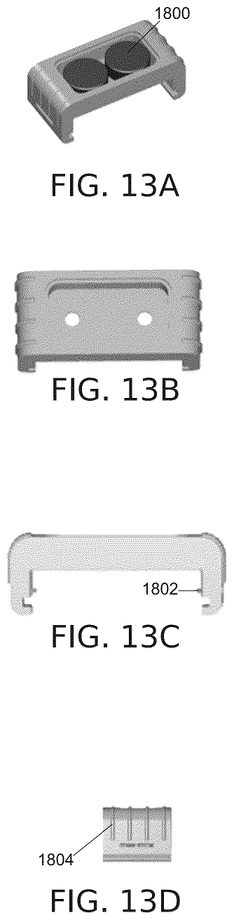

Reference is now made to FIGS. 13A-13D which show various structural features of an actuator, for example as described in FIGS. 12A-12B, according to some embodiments of the present disclosure.

In some embodiments, the actuator is configured for shifting a cover of a source material section using magnetic attraction. Optionally, for example as shown in FIG. 13A, the actuator comprises one or more magnets 1800 sized, positioned and having sufficient magnetic force for shifting a magnetically attracted cover of a source material section. FIG. 13B shows the actuator structure without the magnets.

In some embodiments, for example as shown in FIG. 13C, the actuator comprises electrical connectors 1802 configured to close or become part of an electric circuit of a source material section when the actuator is in position. In some embodiments, closure of the electric circuit activates the heating element so as to heat the source material of the selected section. In some embodiments, the circuit can be closed only once the cover of the selected source material section is shifted. In some embodiments, the circuit can be closed only once airflow commences or is above a given threshold.

In some embodiments, the actuator is shaped and/or sized to be positioned over and optionally across a housing that encases the source material sections. In the example shown herein, the actuator comprises a rectangular profile sized to bridge across the housing. It is noted that the actuator may comprise any other forms suitable for engaging a blocking element of a source material section upon advancement and/or other movement of the actuator, for unblocking the element.

In some embodiments, for example as shown in FIG. 13D, the actuator comprises one or more structural elements shaped for facilitating manual grip of the actuator, such as ribs 1804.

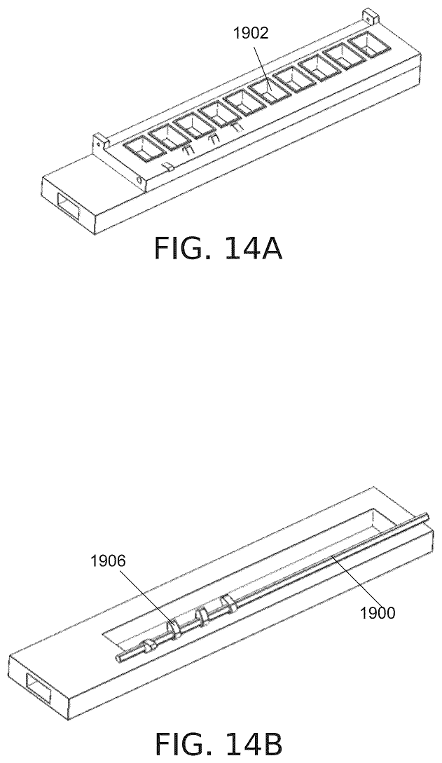

Reference is now made to FIGS. 14A-14B, which illustrate a camshaft mechanism for using a plurality of source material sections in a serial manner, according to some embodiments of the present disclosure.

In some embodiments, a camshaft 1900 extends along at least a portion of the arrayed source material sections 1902 at a position suitable for actuating shifting of the covers of the source material sections (covers are not shown). In some embodiments, lobes 1906 of the camshaft extend to engage the covers so that upon rotation of the camshaft the covers are lifted one after the other by the lobes, thereby allowing air to flow into and through the source material of the opened section.

It is expected that during the life of a patent maturing from this application many relevant decision and processing systems (e.g., AI) will be developed; the scope of the term AI is intended to include all such new technologies a priori.

As used herein with reference to quantity or value, the term “about” means “within ±10% of”.

The terms “comprises”, “comprising”, “includes”, “including”, “having” and their conjugates mean: “including but not limited to”.

The term “consisting of” means: “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

The terms “example”, “exemplary” and “such as” are used herein to mean “serving as an example, instance or illustration”. Any embodiment described as an “example” or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the disclosure may include a plurality of “optional” features except insofar as such features conflict.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Throughout this application, embodiments of this disclosure may be presented with reference to a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination or as suitable in any other described embodiment of the disclosure. Certain features described in the context of various embodiments are not to be considered essential features of those embodiments, unless the embodiment is inoperative without those elements.

What is claimed is:

1. A method of providing feedback to a user of a delivery device configured for administration of at least one active agent, the method comprising:
determining a time since a user initiated a regimen for delivery of the at least one active agent; and
providing to the user a computer interface indication of a current predicted effect of the at least one active agent;
wherein the provided indication is based on input data indicating effects induced in a plurality of users receiving administration of the regimen and the time of said effects since regimen initiation.

2. A system for adjusting an administration regimen based on data gathered during use across a population, the system comprising at least one server configured to:
receive data associated with usage of a delivery device from a plurality of users receiving administration of a regimen using a corresponding plurality of the delivery device, wherein each device is configured to deliver regimen-selectable dosages of one or more active agents from one or more substances, and wherein each delivery device is configured with a data transmission device to transmit to the at least one server input obtained from the user;
determine at least one trend in the received data, based on processing of the data received from the plurality of users;
adjust the regimen based on the at least one trend; and
transmit the adjusted regimen to at least one device for use by at least one user.

3. A system for detection of a potential for substance abuse by a user of a delivery device configured for administration of at least one active agent, the system comprising a server configured to:
receive input data indicating delivery device use histories for a plurality of users of a plurality of delivery devices, wherein the input data include use history data of users previously identified as having transitioned to become a substance abuser; receive input data indicating delivery device use histories for at least one target user;
identify at least one of the target users at potential risk for transitioning to become a substance abuser, based on similarity of the target user's use history to use histories of identified substance abusers, in periods before they became substance abusers.
